(19) United States
(12) Patent Application Publication
(10) Pub. No.: US 2020/0147117 A1
(43) Pub. Date: May 14, 2020

(54) COMBINATION CANCER THERAPY

(71) Applicant: BIOSIGHT LTD., Lod (IL)

(72) Inventors: Ruth BEN YAKAR, Shoham (IL); Stela Gengrinovitch, Kfar Hanania (IL); Shoshi Tessler, Zichron Yaakov (IL); Liat Flaishon, Ramat Gan (IL)

(73) Assignee: BIOSIGHT LTD., Lod (IL)

(21) Appl. No.: 16/738,115

(22) Filed: Jan. 9, 2020

Related U.S. Application Data
(63) Continuation-in-part of application No. PCT/IB2018/000852, filed on Jul. 9, 2018.
(60) Provisional application No. 62/530,213, filed on Jul. 9, 2017.

(51) Int. Cl. A61K 31/7068; A61K 39/395; A61K 31/706; A61K 31/454; A61K 31/439; A61K 31/7084; A61K 31/496; A61K 31/4965; A61K 31/553; A61K 31/5377; A61K 31/4709; A61K 31/704; A61K 9/00; A61P 35/02

(52) U.S. Cl. CPC A61K 31/7068; A61K 39/3955; A61K 31/706; A61K 31/454; A61K 31/439; A61K 31/7084; A61P 35/02; A61K 31/4965; A61K 31/553; A61K 31/5377; A61K 31/4709; A61K 31/704; A61K 9/0019; A61K 31/496

(57) ABSTRACT

The present invention relates to combination therapies of a cytarabine conjugate and one or more anti-neoplastic agents for inhibiting cancer cell growth. In particular, the present invention relates to a conjugate of cytarabine and aspartic acid (BST-236) in combination with one or more additional anti-neoplastic agents for use in the treatment of hematological cancers.

### CROSS REFERENCE TO RELATED APPLICATIONS

This application is a Continuation-in-Part Application of PCT International Patent Application No. PCT/IB2018/000852 filed on Jul. 9, 2018, which claims the benefit of U.S. Provisional Application Ser. No. 62/530,213, filed on Jul. 9, 2017, which are all incorporated in their entirety herein by reference.

### FIELD OF THE INVENTION

The present invention relates to combination therapies of a cytarabine conjugate and one or more additional anti-neoplastic agents for inhibiting cancer cell growth. In particular, the present invention relates to a conjugate of cytarabine and aspartic acid in combination with one or more additional anti-neoplastic agents for use in the treatment of hematological cancers.

### BACKGROUND OF THE INVENTION

### Anti-Neoplastic Agents

Anti-neoplastic agents, also known as anti-proliferative drugs, anti-metabolites or covalent DNA binding drugs, act by inhibiting essential metabolic pathways and are commonly used in the treatment of malignant diseases. However, their high toxicity to normal cells and severe side effects limit their use as therapeutic agents. Undesirable side effects include anemia, emesis and balding due to cytotoxic effects on rapidly dividing normal cells, such as stem cells in the bone marrow, epithelial cells of the intestinal tract, hair follicle cells, etc.

Another major problem associated with anti-proliferative drugs is inherent or acquired resistance of tumors to the drugs. For example, although the initial remission rate following treatment with L-asparaginase is quite high in acute lymphoblastic leukemia (ALL) patients, relapse and associated drug resistance pose a significant clinical problem. Studies have demonstrated increased asparagine synthetase (AS) expression in asparaginase-resistant cells, which has led to the hypothesis that elevated AS activity permits drug-resistant survival of malignant cells.

### Nucleotide/Nucleoside Analogs

Nucleoside analogs compete with their physiologic counterparts for incorporation into nucleic acids and have earned an important place in the treatment of acute leukemia. The most important of these are the arabinose nucleosides; a unique class of anti-metabolites originally isolated from the sponge Cryptothethya crypta, but now produced synthetically. They differ from the physiologic deoxyribonucleosides by the presence of a 2′-OH group in the cis configuration relative to the N-glycosyl bond between cytosine and arabinoside sugar. Several arabinose nucleosides have useful antitumor and antiviral effects. The most active cytotoxic agent of this class is cytosine arabinoside (cytarabine or ara-C). Cytarabine is currently used for treating cancers of white blood cells such as Acute Myeloid Leukemia (AML), Acute Lymphoblastic Leukemia (ALL), Chronic Myeloid Leukemia (CML), Chronic Lymphoblastic Leukemia (CLL), and Myelodysplastic Syndromes (MDS). However, cytarabine is highly toxic having severe side-effects such as cerebellar toxicity and bone marrow suppression. Cytarabine treatment is therefore limited, and often restricted in elderly patients and in patients having hepatic, renal, or cerebellar dysfunction.

One objective of analog development in the area of cytidine antimetabolites has been to find compounds that preserve the inhibitory activity of cytarabine, that are more stable and show higher bioavailability than cytarabine. A number of deaminase-resistant analogs have been developed, including cyclo-cytidine and N⁴-behenoyl ara-C that showed anti-leukemic activity in some clinical trials, but had undesirable side effects. Other representative compounds are cytarabine conjugated with poly-H⁵ (2-hydroxyethyl)-L-glutamine, Dihydro-5-azacitidine, a lipid conjugated derivative of cytarabine designated Elacytarabine, and the amino acid conjugate ValCytarabine (Chhikara et al. Expert. Opin. Drug Deliv. 7: 1399-1414, 2010).

Nucleotide analogs have also been used in non-cancer applications. For example, Flucytosine, a fluorinated cytosine analog, is used as an antifungal agent.

In that side effects associated with cancer treatments in general can be severe and debilitating, there is an unmet need for improved cancer therapies which offer therapeutically effective doses of anti-cancer drugs with limited toxicity and side-effects.

### SUMMARY OF THE INVENTION

The present invention provides combination therapies of a conjugate comprising cytarabine and aspartic acid or a pharmaceutically acceptable salt thereof, and one or more additional anti-neoplastic agents for use in the inhibition of cancer cell growth. The methods of the present invention are particularly useful for reducing cancer cell proliferation, reducing cancer burden, and/or treating hematological cancers.

The present invention is based in part on the unexpected findings that incubation of hematological cancer cells in vitro with a conjugate of aspartic acid and cytarabine, designated herein below Asp-Cytarabine (also referred to herein as Asp-Cyt or BST-236), in which cytarabine is covalently attached to the carboxyl group of the side chain of aspartic acid, together with another anti-neoplastic drug, e.g., the pyrimidine analog azacytidine, resulted in a synergistic inhibitory effect on the proliferation and survival of the hematological cancer cells. Similar effects were obtained with a plurality of hematological cancer cell types.

Further to the above, treatment of an animal model of human leukemia, namely immunocompromised mice into which human leukemia had been introduced, demonstrated that a combination of Asp-Cytarabine and azacytidine exhibited synergistic effects as evidenced by a significant decrease in the number of cancer cells in the spleens (as reflected by a decrease in spleen size) of mice treated with the combination. Mice treated with either Asp-Cytarabine or azacytidine also exhibited reduced spleen weight, but the effect of the combination of the two agents exceeded the additive effect of each in isolation.

Combination therapy comprising administering Asp-Cytarabine and azacytidine is envisioned for treating any subject afflicted with a cancer. In a particular embodiment the cancer is a hematological cancer. In a particular embodiment thereof, combination therapy as disclosed herein is used to treat a subject afflicted with acute lymphocytic leukemia (ALL) or acute myeloid leukemia (AML). In a more particular embodiment, the subject is a medically compromised subject. In even more particular embodiments, the medically compromise subject cannot receive standard cytarabine chemotherapy or other standard chemotherapeutic treatments due to the subject's physical condition and/or known or suspected sensitivity to such treatments. A combination of a high-dose of Asp-Cytarabine and, for example, azacytidine is, therefore, well suited for treating medically compromised subjects because combination therapy such as that described herein has tolerable side-effects and causes less damage to vital organs and tissues. Moreover, the combined treatment of Asp-Cytarabine and azacytidine appears to be effective in prolonging the remission period and the life span of the treated patients as compared to each of the treatments alone.

Thus, the present invention provides effective combination therapies of Asp-Cytarabine with other anti-neoplastic drugs(s) for cancer patients in general, and for medically compromised hematological cancer patients who are typically deprived of standard chemotherapeutic regimen due to their low tolerance for chemotherapy. The present invention therefore fulfils an unmet need in that it presents highly efficacious chemotherapeutic combination treatments for cancer patients and overcomes the impediment of dose limiting toxicities of other combination cancer treatments, including, e.g., cytarabine in combination with other anti-neoplastic drugs.

According to one aspect, a first pharmaceutical composition and a second pharmaceutical composition for use in reducing cancer cell proliferation are presented, wherein the first pharmaceutical composition comprises a therapeutically effective amount of a compound represented by the structure of formula (1):

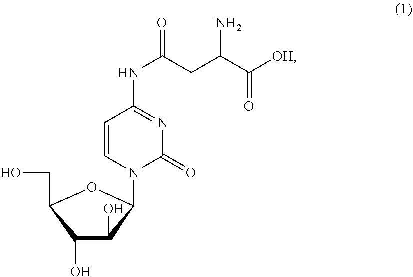

or a pharmaceutically acceptable salt thereof, and
a pharmaceutically acceptable excipient;
and wherein the second pharmaceutical composition comprises a therapeutically effective amount of at least one additional anti-neoplastic agent, wherein the at least one anti-neoplastic agent is a pyrimidine analog, a fms like kinase-3 (FLT-3) inhibitor, a sonic hedgehog inhibitor, an antibody, a drug that target P53 a Bcl-2 inhibitor, an anthracycline, or an isocitrate dehydrogensase (IDH) inhibitor, and
a pharmaceutically acceptable excipient; and
wherein the first and second pharmaceutical compositions are used concurrently or within four hours of each other.

Additionally or alternatively, the above first pharmaceutical composition and the second pharmaceutical composition for use in reducing cancer cell proliferation may include one or more of the following features individually or in combination: wherein the pharmaceutically acceptable salt of the conjugate of formula (1) is a salt of an organic or inorganic acid is acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid or oxalic acid; wherein the pharmaceutically acceptable salt is a salt of acetic acid; wherein the pharmaceutically acceptable salt of the conjugate of formula (1) is a salt of hydrochloric acid; wherein the pyrimidine analog is azacitidine, decitabine, guadecitabine (SGI-110), gemcitabine, or zidovudine; wherein the pyrimidine analog is azacitidine; wherein the Bcl-2 inhibitor is venetoclax (ABT-199); wherein the FLT-3 inhibitor is sorafenib, midostaurin, quizartinib, crenolanib, or gilertinib; wherein the anthracycline is daunorubicin, idarubicin, or doxorubicin; wherein the IDH inhibitor is an IDH1 inhibitor, an IDH2 inhibitor, ivosidenib (AG-120), enasidenib (AG221), IDH305, or FT-2102; wherein the the drug that targets P53 is APR246; wherein the sonic hedgehog inhibitor is glasdegib; wherein the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates; wherein the IDH inhibitor is an IDH1 inhibitor (e.g., AG-120), or an IDH2 inhibitor; wherein the use further comprises use for treating cancer; wherein the cancer is a hematological cancer or a non-hematological cancer; wherein the hematological cancer is a leukemia, a lymphoma, a myeloma or a Myelodysplastic Syndrome (MDS); wherein the leukemia is Acute Myeloid Leukemia (AML), Acute Lymphoblastic Leukemia (ALL), Chronic Myeloid Leukemia (CML), or Chronic Lymphoblastic Leukemia (CLL); wherein the AML is newly diagnosed AML, secondary AML, or relapsed/refractory AML; wherein the lymphoma is Hodgkin's lymphoma or non-Hodgkin's lymphoma; wherein the subject is a mammal; wherein the mammal is a human; wherein the mammal is a medically compromised mammal or the human is a medically compromised human; wherein the medically compromised mammal or human is an elderly mammal or human, a mammal or human having hepatic dysfunction, a mammal or human having renal dysfunction, a mammal or human having pancreatic dysfunction, a mammal or human having bone marrow dysfunction, a mammal or human having cerebellar dysfunction, a mammal or human having an immunological disorder, a mammal or human having refractory or relapsed hematological cancer, or any combination thereof; the elderly human is 70 or more years of age; wherein the pharmaceutical composition comprising the conjugate of formula (1) is administered parenterally; wherein the first pharmaceutical composition is administered intravenously; wherein the conjugate of formula (1) administered to the subject ranges from about 0.3 g/m² to about 6 g/m² of the subject's body surface area per day; wherein the dosage of the conjugate of formula (1) administered to the subject ranges from about 0.8 g/m² to about 6 g/m² of the subject's body surface area per day; wherein the second pharmaceutical composition is administered prior to, concurrent with, or after the first pharmaceutical composition is administered; and/or wherein the second pharmaceutical composition is administered concurrently with the first pharmaceutical composition.

According to another aspect, a pharmaceutical composition for use in reducing cancer cell proliferation is presented, wherein the pharmaceutical composition comprises:

(i) a therapeutically effective amount of a compound represented by the structure of formula (1):

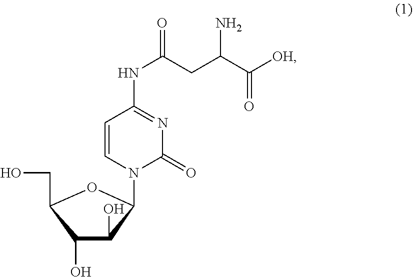

or a pharmaceutically acceptable salt thereof;
(ii) a therapeutically effective amount of an additional anti-neoplastic agent, wherein the at least one anti-neoplastic agent is a pyrimidine analog, a FLT-3 inhibitor, a sonic hedgehog inhibitor, an antibody, a drug that target P53 a Bcl-2 inhibitor, an anthracycline, or an isocitrate dehydrogensase (IDH) inhibitor; and
(iii) a pharmaceutically acceptable excipient.

Additionally or alternatively, the above pharmaceutical composition for use in reducing cancer cell proliferation and comprising (i), (ii), and (iii) may include one or more of the following features individually or in combination: wherein the pharmaceutically acceptable salt of the conjugate of formula (1) is a salt of an organic or inorganic acid, wherein the organic or inorganic acid is acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid, or oxalic acid; wherein the pharmaceutically acceptable salt of the conjugate of formula (1) is a salt of acetic acid; wherein the pharmaceutically acceptable salt of the conjugate of formula (1) is a salt of hydrochloric acid; wherein the pyrimidine analog is azacitidine, decitabine, guadecitabine (SGI-110), gemcitabine, or zidovudine; wherein the pyrimidine analog is azacitidine; wherein the Bcl-2 inhibitor is venetoclax (ABT-199); wherein the FLT-3 inhibitor is sorafenib, midostaurin, quizartinib, crenolanib, or gilertinib; wherein the anthracycline is daunorubicin, idarubicin, or doxorubicin; wherein the IDH inhibitor is an IDH1 inhibitor, an IDH2 inhibitor, AG-120 (ivosidenib), AG221 (enasidenib), IDH305, or FT-2102; wherein the IDH inhibitor is an IDH1 inhibitor, an IDH2 inhibitor, or AG-120 (ivosidenib); wherein the drug that targets P53 is APR246; wherein the sonic hedgehog inhibitor is glasdegib; wherein the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates; wherein reducing cancer cell proliferation further comprises treating a cancer, wherein the cancer is a hematological cancer or a non-hematological cancer; wherein the hematological cancer is a leukemia, a lymphoma, a myeloma or a Myelodysplastic Syndrome (MDS); wherein the leukemia is Acute Myeloid Leukemia (AML), Acute Lymphoblastic Leukemia (ALL), Chronic Myeloid Leukemia (CML), or Chronic Lymphoblastic Leukemia (CLL); wherein the AML is newly diagnosed AML, secondary AML, or relapsed/refractory AML; wherein the lymphoma is Hodgkin's lymphoma or non-Hodgkin's lymphoma; wherein the subject is a mammal; wherein the mammal is a human; wherein the mammal is a medically compromised mammal or the human is a medically compromised human; wherein the medically compromised mammal or human is an elderly mammal or human, a mammal or human having hepatic dysfunction, a mammal or human having renal dysfunction, a mammal or human having pancreatic dysfunction, a mammal or human having bone marrow dysfunction, a mammal or human having cerebellar dysfunction, a mammal or human having an immunological disorder, a mammal or human having refractory or relapsed hematological cancer, or any combination thereof; wherein the elderly human is 70 or more years of age; wherein the pharmaceutical composition for use being administered parenterally; wherein the pharmaceutical composition for use being administered intravenously; wherein the dosage of the conjugate of formula (1) administered to the subject ranges from about 0.3 g/m² to about 6 g/m² of the subject's body surface area per day; wherein the dosage of the conjugate of formula (1) administered to the subject ranges from about 0.8 g/m² to about 6 g/m² of the subject's body surface area per day.

According to another aspect, a method for reducing cancer cell proliferation in a subject afflicted with a cancer is presented, comprising:

(a) administering a therapeutically effective amount of a compound represented by the structure of formula (1):

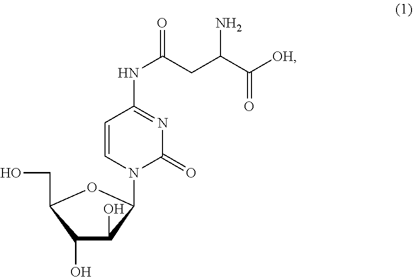

or a pharmaceutically acceptable salt thereof,

or a first pharmaceutical composition comprising the compound of formula (1) or a pharmaceutically acceptable salt thereof; and

(b) administering a therapeutically effective amount of at least one additional anti-neoplastic agent or a second pharmaceutical composition comprising the at least one additional anti-neoplastic agent, wherein the at least one anti-neoplastic agent is a pyrimidine analog, a fms like kinase-3 (FLT-3) inhibitor, a sonic hedgehog inhibitor, an antibody, a drug that target P53, a Bcl-2 inhibitor, an anthracycline, or an isocitrate dehydrogensase (IDH) inhibitor,

wherein the first and second pharmaceutical compositions are administered to the subject concurrently or within four hours of each other, thereby reducing cancer cell proliferation in the subject.

According to another aspect, a method for treating a cancer in a subject afflicted with the cancer, comprising:

(a) administering a therapeutically effective amount of a compound represented by the structure of formula (1):

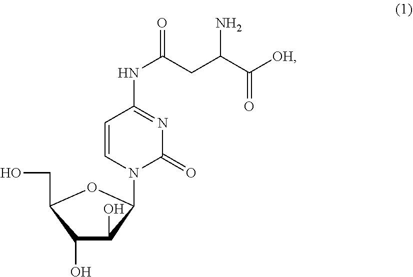

or a pharmaceutically acceptable salt thereof,

or a first pharmaceutical composition comprising the compound of formula (1) or a pharmaceutically acceptable salt thereof; and

(b) administering a therapeutically effective amount of at least one additional anti-neoplastic agent or a second pharmaceutical composition comprising the at least one additional anti-neoplastic agent, wherein the at least one anti-neoplastic agent is a pyrimidine analog, a fms like kinase-3 (FLT-3) inhibitor, a sonic hedgehog inhibitor, an antibody, a drug that target P53, a Bcl-2 inhibitor, an anthracycline, or an isocitrate dehydrogensase (IDH) inhibitor,

wherein the first and second pharmaceutical compositions are administered to the subject concurrently or within four hours of each other, thereby treating the cancer in the subject.

Additionally or alternatively, the above method for reducing cancer cell proliferation or above method for treating a cancer may include one or more of the following features individually or in combination: wherein the second pharmaceutical composition is administered prior to, concomitant with, or after the first pharmaceutical composition is administered; wherein the second pharmaceutical composition is administered concurrently with the first pharmaceutical composition; wherein the pharmaceutically acceptable salt of the conjugate of formula (1) is a salt of an organic or inorganic acid is acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid or oxalic acid; wherein the pharmaceutically acceptable salt is a salt of acetic acid; wherein the pharmaceutically acceptable salt of the conjugate of formula (1) is a salt of hydrochloric acid; wherein the pyrimidine analog is azacitidine, decitabine, guadecitabine (SGI-110), gemcitabine, or zidovudine; wherein the pyrimidine analog is azacitidine; wherein the Bcl-2 inhibitor is venetoclax (ABT-199); wherein the FLT-3 inhibitor is sorafenib, midostaurin, quizartinib, crenolanib, or gilertinib; wherein the anthracycline is daunorubicin, idarubicin, or doxorubicin; wherein the IDH inhibitor is an IDH1 inhibitor, an IDH2 inhibitor, AG-120 (ivosidenib), AG221 (enasidenib), IDH305, or FT-2102; wherein the IDH inhibitor is an IDH1 inhibitor, an IDH2 inhibitor, or AG-120 (ivosidenib); wherein the drug that targets P53 is APR246; wherein the sonic hedgehog inhibitor is glasdegib; wherein the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates; wherein the cancer is a hematological cancer or a non-hematological cancer; wherein the hematological cancer is a leukemia, a lymphoma, a myeloma or a Myelodysplastic Syndrome (MDS); wherein the leukemia is Acute Myeloid Leukemia (AML), Acute Lymphoblastic Leukemia (ALL), Chronic Myeloid Leukemia (CML), or Chronic Lymphoblastic Leukemia (CLL); wherein the AML is newly diagnosed AML, secondary AML, or relapsed/refractory AML; wherein the lymphoma is Hodgkin's lymphoma or non-Hodgkin's lymphoma; wherein the subject is a mammal; wherein the mammal is a human; wherein the mammal is a medically compromised mammal; wherein the human is a medically compromised human; wherein the medically compromised mammal or human is an elderly mammal or human, a mammal or human having hepatic dysfunction, a mammal or human having renal dysfunction, a mammal or human having pancreatic dysfunction, a mammal or human having bone marrow dysfunction, a mammal or human having cerebellar dysfunction, a mammal or human having an immunological disorder, a mammal or human having refractory or relapsed hematological cancer, or any combination thereof; wherein the elderly human is 70 or more years of age; wherein the pharmaceutical composition comprising the conjugate of formula (1) is administered parenterally; wherein the first pharmaceutical composition is administered intravenously; wherein the dosage of the conjugate of formula (1) administered to the subject ranges from about 0.3 g/m² to about 6 g/m² of the subject's body surface area per day; wherein the dosage of the conjugate of formula (1) administered to the subject ranges from about 0.8 g/m² to about 6 g/m² of the subject's body surface area per day.

According to another aspect, a method for reducing cancer cell proliferation in a subject afflicted with the cancer is presented, comprising:

(a) administering a therapeutically effective amount of a compound represented by the structure of formula (1):

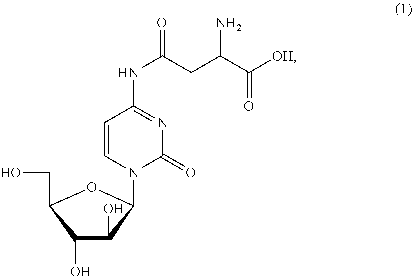

or a pharmaceutically acceptable salt thereof,

or a first pharmaceutical composition comprising the compound of formula (1) or a pharmaceutically acceptable salt thereof; and

(b) administering a therapeutically effective amount of at least one additional anti-neoplastic agent or a second pharmaceutical composition comprising the at least one additional anti-neoplastic agent, wherein the at least one anti-neoplastic agent is a pyrimidine analog, a fms like kinase-3 (FLT-3) inhibitor, a sonic hedgehog inhibitor, an antibody, a drug that target P53, a Bcl-2 inhibitor, an anthracycline, or an isocitrate dehydrogensase (IDH) inhibitor,

wherein the first and second pharmaceutical compositions are administered to the subject concurrently or within four hours of each other, thereby reducing cancer cell proliferation in the subject; and

wherein the administering results in a reduction in side effects in the subject, wherein the side effects comprise at least one of mucositis, diarrhea, or alopecia, relative to side effects observed in subjects treated with cytarabine and the at least one additional anti-neoplastic agent or a second pharmaceutical composition comprising cytarabine and the at least one additional anti-neoplastic agent.

Additionally or alternatively, the above method for reducing cancer cell proliferation, wherein the administering results in a reduction in side effects in the subject, may include one or more of the following features individually or in combination: wherein the second pharmaceutical composition is administered prior to, concomitant with, or after the first pharmaceutical composition is administered; wherein the second pharmaceutical composition is administered concurrently with the first pharmaceutical composition; wherein the pharmaceutically acceptable salt of the conjugate of formula (1) is a salt of an organic or inorganic acid is acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid or oxalic acid; wherein the pharmaceutically acceptable salt is a salt of acetic acid; wherein the pharmaceutically acceptable salt of the conjugate of formula (1) is a salt of hydrochloric acid; wherein the pyrimidine analog is azacitidine, decitabine, guadecitabine (SGI-110), gemcitabine, or zidovudine; wherein the pyrimidine analog is azacitidine; wherein the Bcl-2 inhibitor is venetoclax (ABT-199); wherein the FLT-3 inhibitor is sorafenib, midostaurin, quizartinib, crenolanib, or gilertinib; wherein the anthracycline is daunorubicin, idarubicin, or doxorubicin; wherein the IDH inhibitor is an IDH1 inhibitor, an IDH2 inhibitor, AG-120 (ivosidenib), AG221 (enasidenib), IDH305, or FT-2102; wherein the IDH inhibitor is an IDH1 inhibitor, an IDH2 inhibitor, or AG-120 (ivosidenib); wherein the drug that targets P53 is APR246; wherein the sonic hedgehog inhibitor is glasdegib; wherein the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates; wherein the cancer is a hematological cancer or a non-hematological cancer; wherein the hematological cancer is a leukemia, a lymphoma, a myeloma or a Myelodysplastic Syndrome (MDS); wherein the leukemia is Acute Myeloid Leukemia (AML), Acute Lymphoblastic Leukemia (ALL), Chronic Myeloid Leukemia (CML), or Chronic Lymphoblastic Leukemia (CLL); wherein the AML is newly diagnosed AML, secondary AML, or relapsed/refractory AML; wherein the lymphoma is Hodgkin's lymphoma or non-Hodgkin's lymphoma; wherein the subject is a mammal; wherein the mammal is a human; wherein the mammal is a medically compromised mammal; wherein the human is a medically compromised human; wherein the medically compromised mammal or human is an elderly mammal or human, a mammal or human having hepatic dysfunction, a mammal or human having renal dysfunction, a mammal or human having pancreatic dysfunction, a mammal or human having bone marrow dysfunction, a mammal or human having cerebellar dysfunction, a mammal or human having an immunological disorder, a mammal or human having refractory or relapsed hematological cancer, or any combination thereof; wherein the elderly human is 70 or more years of age; wherein the pharmaceutical composition comprising the conjugate of formula (1) is administered parenterally; wherein the first pharmaceutical composition is administered intravenously; wherein the dosage of the conjugate of formula (1) administered to the subject ranges from about 0.3 g/m² to about 6 g/m² of the subject's body surface area per day; wherein the dosage of the conjugate of formula (1) administered to the subject ranges from about 0.8 g/m² to about 6 g/m² of the subject's body surface area per day.

According to another aspect, the present invention provides a method of inhibiting cancer cell growth in a subject comprising administering to the subject: (a) a pharmaceutical composition comprising a therapeutically effective amount of a conjugate of aspartic acid and cytarabine, designated herein below Asp-Cytarabine, or a pharmaceutically acceptable salt thereof, wherein cytarabine being attached to the aspartic acid through the side chain functional group of said aspartic acid as represented by the structure of formula (1):

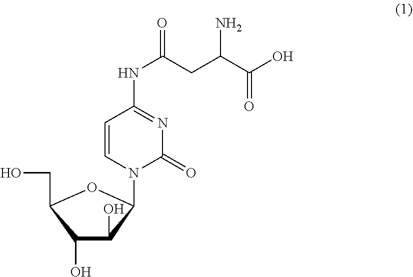

and (b) a pharmaceutical composition comprising a therapeutically effective amount of at least one additional anti-neoplastic agent. In a particular embodiment, the Asp conjugated to cytarabine is an L isomer. In another particular embodiment, the Asp conjugated to cytarabine is a D isomer.

According to some embodiments, the pharmaceutically acceptable salt of Asp-Cytarabine is a salt of an organic or inorganic acid or a residue of an acid. According to additional embodiments, the acid is selected from the group consisting of acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid and oxalic acid. Each possibility represents a separate embodiment of the present invention.

According to one embodiment, the pharmaceutically acceptable salt is a salt of acetic acid. According to another embodiment, the pharmaceutically acceptable salt is a salt of hydrochloric acid (HCl).

According to additional embodiments, the anti-neoplastic agent is a small chemical entity.

According to further embodiments, the small chemical entity is selected from the group consisting of hypomethylating agents/DNA methyltransferase (DNMT) inhibitors, isocitrate dehydrogensase (IDH) inhibitors, histone deacetylase (HDAC) inhibitors, Bromodomain and extraterminal (BET) inhibitors, disruptor of telomeric silencing-1 like (DOT1L) inhibitors, lysine-specific demethylase-1 (LSD1) inhibitors, and Enhancer of zeste homologue 2 (EZH2) inhibitors. Each possibility represents a separate embodiment of the present invention.

According to some embodiments, the hypomethylating agent/DNA methyltransferase (DNMT) inhibitor is a pyrimidine analog selected from the group consisting of azacitidine, decitabine, guadecitabine (SGI-110), gemcitabine, and zidovudine.

According to additional embodiments, the IDH inhibitor is selected from the group consisting of IDH1 inhibitors, IDH2 inhibitors, AG-120 (ivosidenib), AG221 (enasidenib), IDH305, and FT-2102.

According to further embodiments, the HDAC inhibitor is selected from the group consisting of belinostat, panobinostat, vorinostat, entinostat, pracinostat, lenalidomide, and romidepsin.

According to yet further embodiments, the BET inhibitor is selected from the group consisting of OTX015, TEN-010, GSK525762, and CPI-0610.

According to additional embodiments, the DOT1L inhibitor is pinometostat.

According to additional embodiments, the LSD1 inhibitor is selected from the group consisting of tranylcypromide (TCP), GSK2879552, ORY-1001, and IMG-7289.

According to further embodiments, the EZH2 inhibitor is GSK126 or Tazemetostat.

According to additional embodiments, the small chemical entity is selected from the group consisting of anti-metabolites, Bcl-2 inhibitors, anthracyclines, anthracenediones, anti-microtubule agents, alkylating agents, cisplatin and cisplatin analogs, anti-tumor antibiotic agents, topoisomerase inhibitors, thalidomide and thalidomide analogs, angiogenesis inhibitors, proteasome inhibitors, Sonic hedgehog pathway inhibitors, kinase inhibitors, protein translation inhibitors, heat shock protein inhibitors, cytokine pathway inhibitors, telomeric silencing inhibitors, cell cycle inhibitors, murine double minute-2 (Mdm-2) inhibitors, corticosteroids, all-trans retinoic acid, fenretinide, arsenic trioxide, and hydroxyurea. Each possibility represents a separate embodiment of the present invention.

According to further embodiments, the anti-metabolite is selected from the group consisting of pyrimidine analogs, purine analogs, quinolone derivatives, and antifolates.

According to still further embodiments, the pyrimidine analog is selected from the group consisting of azacitidine, decitabine, guadecitabine (SGI-110), gemcitabine, and zidovudine.

According to yet further embodiments, the purine analog is selected from the group consisting of cladribine, clofarabine, fludarabine, nelarabine, pentostatine, 6-mercaptopurine, and ganciclovir.

According to further embodiments, the quinolone derivative is vosaroxin.

According to still further embodiments, the antifolate is selected from the group consisting of methotrexate and pralatrexate.

According to another embodiment, the Bcl-2 inhibitor is venetoclax (ABT-199).

According to another embodiment, the drug that targets P53 is APR246.

According to another embodiment, the sonic hedgehog inhibitor is glasdegib.

According to another embodiment, the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates.

According to yet further embodiments, the anthracycline is selected from the group consisting of daunorubicin, idarubicin, and doxorubicin.

According to another embodiment, the anthracenedione is mitoxantrone.

According to additional embodiments, the anti-microtubule agent is selected from the group consisting of vincristine, vinblastine, and vinorelbine.

According to further embodiments, the alkylating agent is selected from the group consisting of cyclophosphamide, bendamustine, chlorambucil, and ifosfamid.

According to yet further embodiments, the cisplatin analog is selected from the group consisting of oxaliplatin and carboplatin.

According to still further embodiments, the anti-tumor antibiotic agent is selected from the group consisting of cyclosporine, bleomycin, sirolimus (rapamycin), and evarolimus.

According to further embodiments, the topoisomerase inhibitor is selected from the group consisting of etoposide, vosaroxin, and topotecan.

According to further embodiment, the thalidomide analog is selected from the group consisting of lenalidomide and pomalidomide.

According to still further embodiments, the angiogenesis inhibitor is selected from the group consisting of itraconazole, carboxyamidotriazole, angiostatin, endostatin, thalidomide, and lenalidomide.

According to yet further embodiments, the proteasome inhibitor is selected from the group consisting of bortezomib, ixazomib, pevonedistat, carfilzomib, and panobinostat.

According to another embodiment, the Sonic hedgehog pathway inhibitor is glasdegib.

According to yet further embodiments, the kinase inhibitor is selected from the group consisting of tyrosine kinase inhibitors, serine/threonine kinase inhibitors, phosphoinositide kinase inhibitors, and cyclin dependent kinase inhibitors. Each possibility represents a separate embodiment of the invention.

According to still further embodiments, the tyrosine kinase inhibitor is selected from the group consisting of fms-like tyrosine kinase inhibitor 3 (FLT3), growth factor tyrosine kinase inhibitor, Bcr-Abl tyrosine kinase inhibitor, spleen tyrosine kinase inhibitor, janus kinase (jak) inhibitor, bruton's tyrosine kinase inhibitor, and anaplastic lymphoma kinase (Alk) inhibitor. Each possibility represents a separate embodiment of the invention.

According to yet further embodiments, the FLT3 inhibitor is selected from the group consisting of midostaurin, gilteritinib, quizartinib, bortezomib, lestaurtinib, cabozantanib, sunitinib and crenolanib.

According to another embodiment, the growth factor tyrosine kinase inhibitor is sorafenib.

According to some embodiments, the Bcr-Abl tyrosine kinase inhibitor is selected from the group consisting of imatinib (Gleevec), ponatinib, dasatinib, nilotinib, bosutinib, and asciminib .

According to further embodiments, the spleen tyrosine kinase inhibitor is selected from the group consisting of entoplentinib and fostamatinib.

According to other embodiments, the Janus kinase (Jak) inhibitor is selected from the group consisting of tofacitinib, ruxolitinib, oclacitinib, itacitinib, and baricitinib.

According to some embodiments, the bruton's tyrosine kinase inhibitor is selected from the group consisting of ibrutinib, tirabrutinib, and spebrutinib.

According to additional embodiments, the anaplastic lymphoma kinase (Alk) inhibitor is selected from the group consisting of brigatinib, seritinib, crizotinib, and alectinib.

According to further embodiments, the serine/threonine kinase inhibitor is selected from the group consisting of vemurafenib and volasertib.

According to yet further embodiments, the phosphoinositide kinase inhibitor is selected from the group consisting of idalelisib, duvelisib, perifosine, umbralisib, copanlisib, and buparlisib.

According to still further embodiments, the cyclin dependent kinase inhibitor is selected from the group consisting of palbociclib, alvocidib, and dinaciclib.

According to another embodiment, the protein translation inhibitor is omacetaxine.

According to further embodiments, the heat shock protein inhibitor is selected from the group consisting of ganetespid and gamitrinib.

According to further embodiments, the cytokine pathway inhibitor is Ulocuplumab.

According to yet further embodiments, the telomeric silencing inhibitor is EPZ-5676.

According to still further embodiments, the cell cycle inhibitor is p27Kip1.

According to another embodiment, the Mdm-2 inhibitor is idasanutlin.

According to still further embodiments, the corticosteroid is selected from the group consisting of prednisone, dexamethasone, methylprednisolone, and hydrocortisone.

According to some embodiments, the anti-neoplastic agent is a peptide, a protein, or an antibody having anti-neoplastic activity. Each possibility represents a separate embodiment of the invention.

According to further embodiment, the peptide having anti-neoplastic activity is a peptide antibiotic, a peptide antagonist, or a peptidomimetic drug.

According to one embodiment, the peptide antibiotic is bleomycin.

According to another embodiment, the peptide antagonist is BL-8040 (CXCR4 antagonist).

According to a further embodiment, the peptidomimetic drug is TL32711.

According to additional embodiments, the protein having anti-neoplastic activity is selected from the group consisting of cytokines or fusion proteins thereof, interferons or fusion proteins thereof, erythropoietin analogs, and asparaginase.

According to further embodiments, the cytokines, interferons, or fusion protein thereof are selected from the group consisting of granulocyte colony stimulating factor (G-CSF/CSF-3), interferon-alpha, and the fusion protein of interferon, the CD123 inhibitor (e.g., SL-401).

According to another embodiment, the erythropoietin analog is darbepoetin.

According to further embodiments, the antibody having anti-neoplastic activity is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates. Each possibility represents a separate embodiment of the invention.

According to yet further embodiments, the anti CD19 antibody is selected from the group consisting of blinatumomab and coltuximab.

According to still further embodiments, the anti CD20 antibody is selected from the group consisting of rituximab, obinutuzumab, ofatumumab, veltuzumab, ocaratuzumab, and ublituximab.

According to yet further embodiments, the anti CD22 antibody is selected from the group consisting of bepratuzumab and inotuzumab.

According to one embodiment, the anti CD30 antibody is brentuximab.

According to additional embodiment, the anti CD33 antibody is gemtuzumab ozogamicin.

According to a further embodiment, the anti CD37 antibody is otlertuzumab.

According to yet further embodiments, the anti CD38 antibody is selected from the group consisting of daratuzomomab and izatuximab.

According to further embodiments, the anti CD47 antibody is selected from the group consisting of Hu5F9 and CC-90002.

According to yet further embodiment, the anti CD52 antibody is alemtuzumab.

According to yet further embodiment, the anti CD70 antibody is Argx-110.

According to another embodiment, the anti CD79 antibody is polatuzumab.

According to further embodiment, the anti CD80 antibody is galiximab

According to yet further embodiments, the anti CD123 antibody is selected from the group consisting of CSL362 and talacotuzumab.

According to further embodiments, the immune checkpoint inhibitor is selected from the group consisting of anti PD-1 antibodies, anti PD-L1 antibodies, and anti cytotoxic T-lymphocyte-associated protein (CTLA) antibodies.

According to still further embodiments, the anti PD-1 antibody is selected from the group consisting of nivolumab, pembrolizumab, and pidilizumab.

According to yet further embodiments, the anti PD-L1 antibody is selected from the group consisting of durvalumab, and atezolizumab.

According to one embodiment, the anti CTLA antibody is ipilimumab.

According to additional embodiment, the anti CXCR antibody is Ulocuplumab.

According to further embodiments, the anti-growth factor antibody or growth factor receptor antibody is bevacizumab (Avastin) or panitumumab (anti EGFR antibody), respectively.

According to one embodiment, the anti-metalloproteinase antibody is andecaliximab (anti MMP9 antibody)

According to additional embodiment, the anti-selectin antibody is crizanlizumab (anti p-selectin antibody).

According to further embodiments, the antibody-drug conjugate is selected from the group consisting of gemtuzumab-ozogamycin, inotuzumab-ozogamycin, coltuximab-ravtansine, polatuzumab-vedotin, vadastuximab-talirine, and deninotuzumab-mafodotin.

According to some embodiments, the anti-neoplastic agent is bound or attached to immune cells capable of inhibiting cancer cell growth.

According to further embodiments, the immune cells are chimeric antigen receptor T cells (CART). According to one embodiment, the CART is CART123. In another embodiment, CART123 is defined as expressing an anti-CD-123 polypeptide.

A skilled artisan would appreciate that the term “chimeric antigen receptor” or “CAR” may encompass an antigen-binding domain that is fused to an intracellular signaling domain capable of activating or stimulating an immune cell. In one embodiment, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries), in various embodiments, this scFv is fused to a transmembrane domain and then to an intracellular signaling domain. In various embodiments, the CAR is selected to have high affinity or avidity for the antigen. The skilled artisan would recognize that the immune cells disclosed herein expressing CAR directed to specific antigen-binding domains are termed CARTX, wherein “X” represents the antigen target.

In another embodiment, the CART is selected from CART123, CART33, CART 34, CART38, CART56 and CART117.

According to additional embodiments, the method of inhibiting cancer cell growth further comprises treating a cancer selected from the group consisting of hematological cancers and non-hematological cancers.

According to further embodiments, the hematological cancer is selected from the group consisting of leukemias, lymphomas, multiple myeloma, and Myelodysplastic Syndromes (MDS). Each possibility represents a separate embodiment of the invention.

According to yet further embodiments, the leukemia is selected from the group consisting of Acute Myeloid Leukemia (AML), Acute Lymphoblastic Leukemia (ALL), Chronic Myeloid Leukemia (CML), and Chronic Lymphoblastic Leukemia (CLL).

According to still further embodiments, the AML is selected from the group consisting of newly diagnosed AML, secondary AML, and relapsed/refractory AML.

According to further embodiments, the lymphoma is selected from Hodgkin's Lymphoma and Non-Hodgkin's Lymphoma (NHL).

According to some embodiments, the subject is a medically compromised subject who is not amenable to treatment with the standard dose of cytarabine or with other standard chemotherapeutic treatment.

According to some embodiments, the medically compromised subject is selected from the group consisting of elderly subjects, subjects having hepatic dysfunction, subjects having renal dysfunction, subjects having pancreatic dysfunction, subjects having bone marrow dysfunction, subjects having cerebellar dysfunction, subjects having immunologic disorder, subjects having any other organ dysfunction which limits the use of cytarabine, subjects having relapsed or refractory hematological cancers, and any combination thereof. Each possibility represents a separate embodiment of the invention.

According to additional embodiments, the elderly subject is a subject of 70 or more years of age, such as of 75 or 85 or more years of age.

According to further embodiments, the pharmaceutical composition comprising Asp-Cytarabine and the pharmaceutical composition comprising the anti-neoplastic agent each are administered independently by a route selected from the group consisting of parenteral, oral, nasal, topical, transdermal, vaginal, and rectal administration routes.

According to yet further embodiments, the parenteral administration route is selected from the group consisting of intravenous, subcutaneous, intraperitoneal, intramuscular, intradermal, and transdermal administration route. According to one embodiment, the pharmaceutical composition comprising the Asp-Cytarabine is administered intravenously. According to an exemplary embodiment, the pharmaceutical composition comprising Asp-Cytarabine is administered by intravenous infusion. According to another exemplary embodiment, the anti-neoplastic agent is azacytidine being administered orally, subcutaneously or intravenously. According to another exemplary embodiment, the anti-neoplastic agent is ABT-199 administered subcutaneously or intravenously.

According to yet further embodiments, Asp-Cytarabine is administered in a daily dose ranging from about 0.3 g/m² to about 10 g/m² of the subject's body surface area, such as a daily dose of about 0.3 g/m², 0.5 g/m², 0.8 g/m², 1 g/m², 1.5 g/m², 2 g/m², 2.3 g/m², 2.5 g/m², 3 g/m², 3.5 g/m², 4 g/m², 4.5 g/m², or 6 g/m² of the subject's surface area or any dose in-between. Each possibility represents a separate embodiment of the invention.

According to additional embodiments, the pharmaceutical composition comprising Asp-Cytarabine is administered once a day for at least 3 days, such as for 4 days, 5, 6, 8, 10, 12, or for 15 consecutive days or any integer in-between. According to further embodiments, the pharmaceutical composition comprising Asp-Cytarabine is administered once a day for 6 consecutive days once or twice a month.

According to yet further embodiments, the pharmaceutical composition comprising Asp-Cytarabine is administered once every other day for at least one week, at least two weeks, three weeks or at least one month.

According to further embodiments, the pharmaceutical composition comprising the additional anti-neoplastic agent is administered once or twice a day for at least 3 days, such as for 4 days, 5, 6, 8, 10, 12, or 20 consecutive days or any integer in between.

According to yet further embodiments, the pharmaceutical composition comprising the additional anti-neoplastic agent is administered once or twice a day for 3 to 15 consecutive days once or twice a month.

According to additional embodiments, the pharmaceutical composition comprising the additional anti-neoplastic agent is administered prior to, concomitant with, and/or after administering the pharmaceutical composition comprising Asp-Cytarabine.

According to another aspect, the present invention provides a method of inhibiting cancer cell growth in a subject comprising administering to the subject a pharmaceutical composition comprising: (i) a therapeutically effective amount of a conjugate of aspartic acid and cytarabine, designated herein Asp-Cytarabine, or a pharmaceutically acceptable salt thereof, wherein cytarabine being attached to the aspartic acid through the side chain functional group of said aspartic acid as represented by the structure of formula (1),

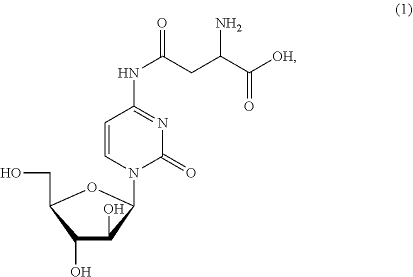

and (ii) a therapeutically effective amount of at least one additional anti-neoplastic agents.

According to some embodiments, the pharmaceutically acceptable salt of Asp-Cytarabine is a salt of an organic or inorganic acid or residue of an acid. According to additional embodiments, the acid is selected from the group consisting of acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid and oxalic acid. According to a certain embodiment, the pharmaceutically acceptable salt is a salt of acetic acid. According to another embodiment, the pharmaceutically acceptable salt is a salt of hydrochloric acid.

According to additional embodiments, the anti-neoplastic agent is a small chemical entity.

According to further embodiments, the small chemical entity is selected from the group consisting of hypomethylating agents/DNA methyltransferase (DNMT) inhibitors, isocitrate dehydrogensase (IDH) inhibitors, histone deacetylase (HDAC) inhibitors, Bromodomain and extraterminal (BET) inhibitors, disruptor of telomeric silencing-1 like (DOT1L) inhibitors, lysine-specific demethylase-1 (LSD1) inhibitors, and Enhancer of zeste homologue 2 (EZH2) inhibitors. Each possibility represents a separate embodiment of the present invention.

According to some embodiments, the hypomethylating agent/DNA methyltransferase (DNMT) inhibitor is a pyrimidine analog selected from the group consisting of azacitidine, decitabine, guadecitabine (SGI-110), gemcitabine, and zidovudine.

According to additional embodiments, the IDH inhibitor is selected from the group consisting of IDH1 inhibitors, IDH2 inhibitors, AG-120 (ivosidenib), AG221 (enasidenib), IDH305, and FT-2102.

According to further embodiments, the HDAC inhibitor is selected from the group consisting of belinostat, panobinostat, vorinostat, entinostat, pracinostat, lenalidomide, and romidepsin.

According to yet further embodiments, the BET inhibitor is selected from the group consisting of OTX015, TEN-010, GSK525762, and CPI-0610.

According to additional embodiments, the DOT1L inhibitor is pinometostat.

According to additional embodiments, the LSD1 inhibitor is selected from the group consisting of tranylcypromide (TCP), GSK2879552, ORY-1001, and IMG-7289.

According to further embodiments, the EZH2 inhibitor is GSK126 or Tazemetostat.

According to additional embodiments, the small chemical entity is selected from the group consisting of anti-metabolites, Bcl-2 inhibitors, anthracyclines, anthracenediones, anti-microtubule agents, alkylating agents, cisplatin and cisplatin analogs, anti-tumor antibiotic agents, topoisomerase inhibitors, thalidomide and thalidomide analogs, angiogenesis inhibitors, proteasome inhibitors, Sonic hedgehog pathway inhibitors, kinase inhibitors, protein translation inhibitors, heat shock protein inhibitors, cytokine pathway inhibitors, telomeric silencing inhibitors, cell cycle inhibitors, murine double minute-2 (Mdm-2) inhibitors, corticosteroids, all-trans retinoic acid, fenretinide, arsenic trioxide, and hydroxyurea. Each possibility represents a separate embodiment of the present invention.

According to further embodiments, the anti-metabolite is selected from the group consisting of pyrimidine analogs, purine analogs, quinolone derivatives, and antifolates.

According to still further embodiments, the pyrimidine analog is selected from the group consisting of azacitidine, decitabine, guadecitabine (SGI-110), gemcitabine, and zidovudine.

According to yet further embodiments, the purine analog is selected from the group consisting of cladribine, clofarabine, fludarabine, nelarabine, pentostatine, 6-mercaptopurine, and ganciclovir.

According to further embodiments, the quinolone derivative is vosaroxin.

According to still further embodiments, the antifolate is selected from the group consisting of methotrexate and pralatrexate.

According to another embodiment, the Bcl-2 inhibitor is venetoclax (ABT-199).

According to yet further embodiments, the anthracycline is selected from the group consisting of daunorubicin, idarubicin, and doxorubicin.

According to another embodiment, the drug that targets P53 is APR246.

According to another embodiment, the sonic hedgehog inhibitor is glasdegib.

According to another embodiment, the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates.

According to another embodiment, the anthracenedione is mitoxantrone.

According to additional embodiments, the anti-microtubule agent is selected from the group consisting of vincristine, vinblastine, and vinorelbine.

According to further embodiments, the alkylating agent is selected from the group consisting of cyclophosphamide, bendamustine, chlorambucil, and ifosfamid.

According to yet further embodiments, the cisplatin analog is selected from the group consisting of oxaliplatin and carboplatin.

According to still further embodiments, the anti-tumor antibiotic agent is selected from the group consisting of cyclosporine, bleomycin, sirolimus (rapamycin), and evarolimus.

According to further embodiments, the topoisomerase inhibitor is selected from the group consisting of etoposide, vosaroxin, and topotecan.

According to further embodiment, the thalidomide analog is selected from the group consisting of lenalidomide and pomalidomide.

According to still further embodiments, the angiogenesis inhibitor is selected from the group consisting of itraconazole, carboxyamidotriazole, angiostatin, endostatin, thalidomide, and lenalidomide.

According to yet further embodiments, the proteasome inhibitor is selected from the group consisting of bortezomib, ixazomib, pevonedistat, carfilzomib, and panobinostat.

According to another embodiment, the Sonic hedgehog pathway inhibitor is glasdegib.

According to yet further embodiments, the kinase inhibitor is selected from the group consisting of tyrosine kinase inhibitors, serine/threonine kinase inhibitors, phosphoinositide kinase inhibitors, and cyclin dependent kinase inhibitors. Each possibility represents a separate embodiment of the invention.

According to still further embodiments, the tyrosine kinase inhibitor is selected from the group consisting of fms-like tyrosine kinase inhibitor 3 (FLT3), growth factor tyrosine kinase inhibitor, Bcr-Abl tyrosine kinase inhibitor, spleen tyrosine kinase inhibitor, janus kinase (jak) inhibitor, bruton's tyrosine kinase inhibitor, and anaplastic lymphoma kinase (Alk) inhibitor. Each possibility represents a separate embodiment of the invention.

According to yet further embodiments, the FLT3 inhibitor is selected from the group consisting of midostaurin, gilteritinib, quizartinib, bortezomib, lestaurtinib, cabozantanib, sunitinib and crenolanib.

According to another embodiment, the growth factor tyrosine kinase inhibitor is sorafenib.

According to some embodiments, the Bcr-Abl tyrosine kinase inhibitor is selected from the group consisting of imatinib (Gleevec), ponatinib, dasatinib, nilotinib, bosutinib, and asciminib.

According to further embodiments, the spleen tyrosine kinase inhibitor is selected from the group consisting of entoplentinib and fostamatinib.

According to other embodiments, the Janus kinase (Jak) inhibitor is selected from the group consisting of tofacitinib, ruxolitinib, oclacitinib, itacitinib, and baricitinib.

According to some embodiments, the bruton's tyrosine kinase inhibitor is selected from the group consisting of ibrutinib, tirabrutinib, and spebrutinib.

According to additional embodiments, the anaplastic lymphoma kinase (Alk) inhibitor is selected from the group consisting of brigatinib, seritinib, crizotinib, and alectinib.

According to further embodiments, the serine/threonine kinase inhibitor is selected from the group consisting of vemurafenib and volasertib.

According to yet further embodiments, the phosphoinositide kinase inhibitor is selected from the group consisting of idalelisib, duvelisib, perifosine, umbralisib, copanlisib, and buparlisib.

According to still further embodiments, the cyclin dependent kinase inhibitor is selected from the group consisting of palbociclib, alvocidib, and dinaciclib.

According to another embodiment, the protein translation inhibitor is omacetaxine.

According to further embodiments, the heat shock protein inhibitor is selected from the group consisting of ganetespid and gamitrinib.

According to further embodiments, the cytokine pathway inhibitor is Ulocuplumab.

According to yet further embodiments, the telomeric silencing inhibitor is EPZ-5676.

According to still further embodiments, the cell cycle inhibitor is p27Kip1.

According to another embodiment, the Mdm-2 inhibitor is idasanutlin.

According to still further embodiments, the corticosteroid is selected from the group consisting of prednisone, dexamethasone, methylprednisolone, and hydrocortisone.

According to some embodiments, the anti-neoplastic agent is a peptide, a protein, or an antibody having anti-neoplastic activity. Each possibility represents a separate embodiment of the invention.

According to further embodiment, the peptide having anti-neoplastic activity is a peptide antibiotic, a peptide antagonist, or a peptidomimetic drug.

According to one embodiment, the peptide antibiotic is bleomycin.

According to another embodiment, the peptide antagonist is BL-8040 (CXCR4 antagonist).

According to a further embodiment, the peptidomimetic drug is TL32711.

According to additional embodiments, the protein having anti-neoplastic activity is selected from the group consisting of cytokines or fusion proteins thereof, interferons or fusion proteins thereof, erythropoietin analogs, and asparaginase.

According to further embodiments, the cytokines, interferons, or fusion protein thereof are selected from the group consisting of granulocyte colony stimulating factor (G-CSF/CSF-3), interferon-alpha, and the fusion protein of interferon, the CD123 inhibitor (e.g., SL-401).

According to another embodiment, the erythropoietin analog is darbepoetin.

According to further embodiments, the antibody having anti-neoplastic activity is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates. Each possibility represents a separate embodiment of the invention.

According to yet further embodiments, the anti CD19 antibody is selected from the group consisting of blinatumomab and coltuximab.

According to still further embodiments, the anti CD20 antibody is selected from the group consisting of rituximab, obinutuzumab, ofatumumab, veltuzumab, ocaratuzumab, and ublituximab.

According to yet further embodiments, the anti CD22 antibody is selected from the group consisting of bepratuzumab and inotuzumab.

According to one embodiment, the anti CD30 antibody is brentuximab.

According to additional embodiment, the anti CD33 antibody is gemtuzumab-ozogamicin.

According to a further embodiment, the anti CD37 antibody is otlertuzumab.

According to yet further embodiments, the anti CD38 antibody is selected from the group consisting of daratuzomomab and izatuximab.

According to further embodiments, the anti CD47 antibody is selected from the group consisting of Hu5F9 and CC-90002.

According to yet further embodiment, the anti CD52 antibody is alemtuzumab.

According to another embodiment, the anti CD79 antibody is polatuzumab.

According to further embodiment, the anti CD80 antibody is galiximab.

According to yet further embodiments, the anti CD123 antibody is selected from the group consisting of CSL362, IMGN632 and talacotuzumab.

According to further embodiments, the immune checkpoint inhibitor is selected from the group consisting of anti PD-1 antibodies, anti PD-L1 antibodies, and anti cytotoxic T-lymphocyte-associated protein (CTLA) antibodies.

According to still further embodiments, the anti PD-1 antibody is selected from the group consisting of nivolumab, pembrolizumab, and pidilizumab.

According to yet further embodiments, the anti PD-L1 antibody is selected from the group consisting of durvalumab, and atezolizumab.

According to one embodiment, the anti CTLA antibody is ipilimumab.

According to additional embodiment, the anti CXCR antibody is Ulocuplumab.

According to further embodiments, the anti-growth factor antibody or growth factor receptor antibody is bevacizumab (Avastin) or panitumumab (anti EGFR antibody), respectively.

According to one embodiment, the anti-metalloproteinase antibody is andecaliximab (anti MMP9 antibody)

According to additional embodiment, the anti-selectin antibody is crizanlizumab (anti p-selectin antibody).

According to further embodiments, the antibody-drug conjugate is selected from the group consisting of gemtuzumab-ozogamycin, inotuzumab-ozogamycin, coltuximab-ravtansine, polatuzumab-vedotin, vadastuximab-talirine, and deninotuzumab-mafodotin.

According to some embodiments, the anti-neoplastic agent is bound or attached to immune cells capable of inhibiting cancer cell growth.

According to further embodiments, the immune cells are chimeric antigen receptor T cells (CART). According to one embodiment, the CART is CART123,CART33, CART34, CART38, CART56 or CART117.

According to additional embodiments, the method of inhibiting cancer cell growth further comprises treating a cancer selected from the group consisting of hematological cancers and non-hematological cancers.

According to further embodiments, the hematological cancer is selected from the group consisting of leukemias, lymphomas, multiple myeloma, and Myelodysplastic Syndromes (MDS).

According to yet further embodiments, the leukemia is selected from the group consisting of Acute Myeloid Leukemia (AML), Acute Lymphoblastic Leukemia (ALL), Chronic Myeloid Leukemia (CML), and Chronic Lymphoblastic Leukemia (CLL).

According to still further embodiments, the AML is selected from the group consisting of newly diagnosed AML, secondary AML, and relapsed/refractory AML.

According to further embodiments, the lymphoma is selected from Hodgkin's Lymphoma and Non-Hodgkin's Lymphoma (NHL).

According to some embodiments, the subject is a medically compromised subject who is not amenable to treatment with the standard dose of cytarabine or with other standard chemotherapeutic treatment.

According to further embodiments, the medically compromised subject is selected from the group consisting of elderly subjects, subjects having hepatic dysfunction, subjects having renal dysfunction, subjects having pancreatic dysfunction, subjects having bone marrow dysfunction, subjects having cerebellar dysfunction, subjects having immunologic disorder, subjects having any other organ dysfunction which limits the use of cytarabine, subjects having relapsed or refractory hematological cancers, and any combination thereof.

According to additional embodiments, the elderly subject is a subject of 70 or more years of age, such as of 75, 80, or 85 or more years of age.

According to yet further embodiments, Asp-Cytarabine is administered in a daily dose ranging from about 0.3 g/m² to about 10 g/m² of the subject's body surface area, such as a daily dose of about 0.3 g/m², 0.5 g/m², 0.8 g/m², 1 g/m², 1.5 g/m², 2 g/m², 2.3 g/m², 2.5 g/m², 3 g/m², 3.5 g/m², 4 g/m², 4.5 g/m², or 6 g/m² of the subject's surface area or any dose in-between.

According to still further embodiments, the pharmaceutical composition is administered parenterally. According to further embodiments, the pharmaceutical composition is administered by intravenous, intraperitoneal, intramuscular, subcutaneous, intrathecal, intradermal, transdermal, or intravesicular administration route. According to a certain embodiment, the pharmaceutical composition is administered intravenously, preferably by infusion.

According to additional embodiments, the pharmaceutical composition is administered once a day for at least 3 days, such as for 4 days, 5, 6, 8, 10, 12, or for 20 consecutive days or any integer in-between. According to further embodiments, the pharmaceutical composition is administered once a day for 6 consecutive days once or twice a month. According to yet further embodiments, the pharmaceutical composition is administered once every other day for at least one week, at least two weeks, three weeks or at least one month.

According to another aspect, the present invention provides a first pharmaceutical composition and a second pharmaceutical composition for use in inhibiting cancer cell growth, wherein the first pharmaceutical composition comprises a therapeutically effective amount of a conjugate of aspartic acid and cytarabine, or a pharmaceutically acceptable salt thereof, wherein cytarabine being attached to the aspartic acid through the side chain functional group of said aspartic acid as represented by the structure of formula (1):

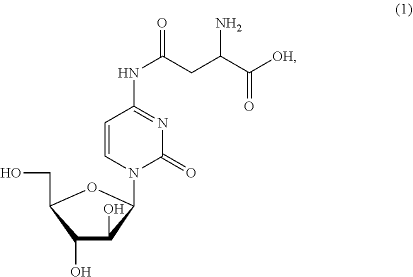

and wherein the second pharmaceutical composition comprises a therapeutically effective amount of at least one additional anti-neoplastic agent according to the principles of the present invention.

According to another aspect, the present invention provides a pharmaceutical composition for use in inhibiting cancer cell growth, the pharmaceutical composition comprises: (i) a therapeutically effective amount of a conjugate of aspartic acid and cytarabine of formula (1), or a pharmaceutically acceptable salt thereof; and (ii) a therapeutically effective amount of an additional anti-neoplastic agent according to the principles of the present invention.

These and other embodiments of the present invention will become apparent in conjunction with the figures, description and claims that follow.

### BRIEF DESCRIPTION OF THE FIGURES

FIG. 1 shows of the inhibitory effect of a combination of Asp-Cytarabine (Asp-Cyt) and Azacytidine (AZA) on the proliferation and survival of U937 cells in vitro.

FIG. 2 shows the inhibitory effect of a combination of Asp-Cytarabine (Asp-Cyt) and Azacytidine (AZA) on the proliferation of Molt-4 cells in vitro.

FIG. 3 shows the inhibitory effect of a combination of Asp-Cytarabine (Asp-Cyt) and ABT-199 (ABT) on the proliferation and survival of U937 cells in vitro.

FIG. 4 shows in vivo data of the combination of BST-236 (BST; Asp-Cytarabine) and Azacytidine (AZA).

### DETAILED DESCRIPTION OF THE INVENTION

The present invention provides methods of reducing cancer cell proliferation in a subject comprising administering to the subject a conjugate of cytarabine covalently linked to aspartic acid (referred to herein as BST-236, Asp-Cytarabine, or Asp-Cyt) in combination with one or more additional anti-neoplastic agents. The methods of the present invention are particularly useful for treating cancer in a subject in need thereof. The synergistic effect of BST-236 in combination with one or more additional anti-neoplastic agent confers a therapeutic benefit to subjects treated with a combination thereof, thereby providing improved therapeutic regimens for such subjects with improved outcomes with respect to morbidity and/or mortality.

In a particular embodiment, the subject is a medically compromised subject. Such subjects/patients typically cannot be treated with the non-conjugated high-dose cytarabine in combination with other anti-neoplastic agents due to severe adverse effects, and thus are given low dose cytarabine which is not sufficiently effective, or given supportive therapy only.

Accordingly, in this embodiment, the present invention fulfills a long-felt need for treating medically compromised patients who have been diagnosed as having hematological cancers, yet cannot be treated with high-dose cytarabine. The conjugates of the present invention enable combination therapies of these cancer patients with cytarabine at doses that would have been toxic if administered in its non-conjugated form, specifically if combined with one, two or more additional anti-neoplastic agents.

### Amino Acids and Proliferative Diseases

Asparagine is a non-essential amino acid that is required by rapidly proliferating cells. Mammalian cells can synthesize asparagine from aspartate using the ATP-dependent enzyme asparagine synthetase (EC 6.3.5.4), which transfers the amino group from the amide of glutamine to the β-carboxyl of aspartate in a reaction that can be represented as: Glutamine+Aspartate+ATP+H₂O=Glutamate+Asparagine+AMP+PPi.

Malignant cells often require higher amounts of amino acids, including asparagine, to support their metabolism and proliferation. In order to fulfill the need for high amounts of amino acids, malignant cells develop the ability to actively transport amino acids from their environment. Moreover, asparagine synthetase deficiency occurs in certain tumors, causing them to rely on an external supply of asparagine from other sources, such as serum. This observation led to the development of the enzyme L-asparaginase (CE 3.5.1.1) as a chemotherapeutic agent. L-asparaginase hydrolyzes L-asparagine to aspartate and ammonia, hence depleting L-asparagine from the serum and inhibiting tumor growth. L-asparaginase is used mainly in the treatment of Acute Lymphoblastic Leukemia (ALL) and shows some activity against other hematological cancers including acute non-lymphocytic leukemia.

The L-asparaginase used in the clinic is available in two unmodified (native) forms purified from bacterial sources, and in one modified form as a PEGylated compound. U.S. Pat. No. 4,179,337 teaches PEGylated L-asparaginase, wherein the enzyme is coupled to PEG having a molecular weight of about 500 to 20,000 Daltons.

The in vivo down-regulation of asparagine synthetase may provide an efficient mechanism for inhibiting tumor growth. However, cells respond to amino acid deprivation by a concerted increase in asparagine synthetase mRNA, protein, and enzymatic activity that involves transcriptional control of the asparagine synthetase gene.

International Patent Application Publication No. WO 2005/072061 to some of the inventors of the present invention discloses compounds comprising a drug covalently linked to a functional group of an amino acid side chain, such compounds are useful for targeting drugs to neoplastic cells.

International Patent Application Publication No. WO 2017/094011 to some of the inventors of the present invention discloses pharmaceutically acceptable salts of conjugates of a cytotoxic, cytostatic or chemotherapeutic agent, such as cytidine analog drugs, and an amino acid, preferably an aspartic acid, glutamic acid, asparagine or glutamine, and use thereof for the treatment of cancer.

International Application Publication No. WO 2017/093993 to some of the inventors of the present invention discloses conjugates of cytarabine and an amino acid selected from the group consisting of aspartic acid, glutamic acid, asparagine and glutamine for use in the treatment of cancer in medically compromised patients.

### Combination Therapy in Cancer

Most of the anti-cancer drugs are typically administered in a combination therapy with other anti-cancer drugs and not as a stand-alone treatment.

In the treatment of AML, a standard dose of cytarabine (100-200 mg/m² of body surface) is administered for seven days in combination with daunorubicin (50-60 mg/m²) or idarubicin for three days. This standard regimen can be combined with oral administration of midostaurine (50 mg/m²) every 12 hours, from the 8^{th} day until the 21^{st} day, or with cladribine (5 mg/m²) for five days or with all-trans retinoic acid (ATRA; 45 mg/m²) for fifteen days. Cytarabine (100-200 mg/m²) administered for seven days can also be administered in combination with mitoxantrone (12 mg/m²) for three days. In AML consolidation when high doses of cytarabine (2 g/m² for patients of the age <50; and 1.5 g/m² for patients of the age 50-60 years) are administered every 12 hours for five days, the combination is limited to a dose of 45 mg/m² of daunorubicin for three days (NCCN Guidelines, Acute Myeloid, Version 3.2017).

For medically fit older patients (>60 years of age), the NCCN recommends treatment with a combination of an anthracycline and a standard dose cytarabine, while for medically unfit older patients who are in poor physical condition or having liver, heart, or kidney dysfunction, the NCCN recommends less intensive chemotherapy with DNA hypomethylating agents (e.g., azacitidine, decitabine), namely administering low-dose cytarabine, or supportive care only.

Treatment of ALL patients includes tyrosine kinase inhibitors (TKIs), such as ponatinib, imatinib, or dasatinib, in combination with hyper-CVAD (cyclophosphamide, vincristine, doxorubicin, and dexamethasone) alternating with high-dose methotrexate and cytarabine. Other combination regimens for ALL can include idarubicin, dexamethasone, vincristine, cyclophosphamide, and cytarabine, optionally with rituximab immunotherapy (NCCN Guidelines, Acute Lymphoblastic Leukemia, Version 1.2017).

B-cell Lymphoma first-line regimen includes combination of cyclophosphamide, doxorubicin, vincristine and prednisone (CHOP regimen) with rituximab. Aggressive first line regimen includes hyper-CVAD alternating with high-dose methotrexate, cytarabine and rituximab Second-line therapy induction regimens may include etoposide, cytarabine, and rituximab, or dexamethasone, cisplatin, cytarabine, and rituximab (NCCN Guidelines, B-cell Lymphoma, Version 3.2017). T-cell Lymphoma preferred first-line regimens are CHOP, CHOEP (including etoposide) alternating with hyper-CVAD or with high-dose methotrexate and cytarabine. Second-line treatment is dexamethasone, cisplatin, and cytarabine (NCCN Guidelines, T-cell Lymphoma, Version 2.2017).

Although a variety of drug combinations for the treatment of cancer are available, the effective combinations are limited due to low efficacy, dose limiting toxicities and drug-drug interactions.

Thus, there is an unmet need for improved cancer combination therapies which enable administering therapeutically effective doses of anti-cancer drugs with limited toxicity and side-effects.

### Definitions

For convenience and clarity certain terms employed in the specification, examples, and claims are described herein.

The term “non-conjugated cytarabine” as used herein refers to cytarabine which is free and not covalently attached to an amino acid. Non-conjugated cytarabine is designated throughout the specification and claims as “cytarabine”.

Treatment with cytarabine is known as “intensive” treatment. The term “intensive” treatment with cytarabine means treatment with a “standard dose” of cytarabine, and optionally with a “high dose” of cytarabine, which refer to 100-200 mg/m²/day, and ≥1 g/m²/day, respectively. Typically, young and medically fit adult patients (18-75 years of age) are treated with the standard dose (100-200 mg/m²/day) of cytarabine. High cytarabine doses (≥1 g/m²) may also be administered to medically fit patients, either as induction or consolidation therapy. However, due to the high toxicity of cytarabine, most of the subjects of 75 or more years of age cannot be treated with the intensive treatment of cytarabine and can be treated with a daily dose of cytarabine of 20 mg/m² of the subject's surface area (known as “low-dose” cytarabine). Some subjects of 75 or more years of age do not benefit from cytarabine treatment at all due to its severe adverse events.

The term “medically compromised” subjects as used herein refers to a sub-population of subjects who are elderly and/or are weakened or impaired medically so that they cannot tolerate the high dose (≥1 g/m²/day) or even the standard dose (100-200 mg/m²/day) of cytarabine (intensive therapy) due to its severe adverse events. Therefore, these subjects are typically treated with low-dose cytarabine (20 mg/m²/day). According to some embodiments, the medically compromised subjects cannot tolerate the non-conjugated cytarabine at all. Medically compromised subjects include, but are not limited to, subjects suffering from or having renal dysfunction, hepatic dysfunction, pancreatic dysfunction, bone marrow dysfunction, cerebellar, dysfunction, immunologic disorder, any other organ, tissue or system dysfunction which limits the use of cytarabine, and a combination thereof, due to its severe side events.

Each possibility disclosed throughout the specification of the present invention represents a separate embodiment of the invention.

The term “elderly subjects” as used herein refers to subjects of 70 years of age or more, and more particularly of 75 years of age or more.

The terms “renal dysfunction”, “hepatic dysfunction”, “pancreatic dysfunction”, “bone marrow dysfunction” and “cerebellar dysfunction” refer to a state in which the organ/tissue function, e.g., kidney, liver, pancreas, bone marrow, and cerebellum, is decreased relative to that of a healthy individual (normal/control state). In general, organ/tissue dysfunction is a state characterized in that any one or more measurement values of inspection items for organ function deviate from the range of normal values (reference values) determined for healthy individuals.

It is to be understood that the side events caused by cytarabine are in some embodiments more severe when using combination therapies of cytarabine with additional anti-neoplastic drug(s).

The phrases “combination therapy” and “combination treatment” as used herein refer to the use of two or more kinds of therapies. Different kinds of therapies may be used in sequence, at the same time, or in various timing formats. Therapy includes chemotherapy, radiation therapy and/or surgery. According to the principles of the present invention, a combination therapy refers to administration of Asp-Cytarabine and any other anti-neoplastic drug. A combination of Asp-Cytarabine and cytarabine is, however, excluded from combination therapy described herein.

The term “dose limiting toxicity” is defined in accordance with the Common Terminology Criteria of Adverse Events Version 3.0 (CTCAE). Dose limiting toxicity occurs upon administration of a compound to a subject if any of the following events are observed within a drug treatment cycle: Grade 4 neutropenia (i.e., absolute neutrophil count (ANC) ≤500 cells/mm³) for 5 or more consecutive days or febrile neutropenia (i.e., fever ≤38.5° C. with an ANC≤1000 cells/mm³); Grade 4 thrombocytopenia (i.e., ≤25,000 cells/mm³ or bleeding episode requiring platelet transfusion); Grade 4 fatigue, or a two-point decline in ECOG performance status; Grade 3 or greater nausea, diarrhea, vomiting, and/or myalgia despite the use of adequate/maximal medical intervention; Grade 3 or greater non-hematological toxicity (except fatigue); retreatment delay of more than 2 weeks due to delayed recovery from toxicity related to treatment with the compound; Grade 2 or greater cardiac toxicity of clinical significance (e.g., a decline in the resting ejection fraction to 40%−≤50% or shortening fraction to 15%−≤24%; cardiac troponin T≥0.05 ng/mL).

The term “cancer” refers to the physiological condition in mammals in which a population of cells is characterized by unregulated cell growth. Examples of cancer include, but are not limited to, leukemia, lymphoma, carcinoma, blastoma, and sarcoma.

“Tumor” and “neoplasm” refer to any mass of tissue that result from excessive cell growth or proliferation, either benign (noncancerous) or malignant (cancerous) including pre-cancerous lesions.

The terms “cancer cell” or “tumor cell” refer to the total population of cells derived from a tumor or a pre-cancerous lesion, which comprise the bulk of the tumor cell population, and tumorigenic stem cells (cancer stem cells).

The terms “inhibiting cancer cell growth” or “inhibiting proliferation of cancer cells” or “inhibiting cancer cell survival” which are interchangeable throughout the specification and claims refer to the capability to prevent, reduce, or arrest the growth, proliferation and/or survival of a cancer cell, a neoplasm or a tumor. Thus, inhibition of cancer cell growth is defined as a reduction in cancer cell growth by at least 10%, or by at least 20%, 30%, 40%, 50%, 60%, 70%, 80%, 90%, or preferably by 100%, as compared to cancer cell growth in the absence of a therapeutic agent or combination therapy comprising Asp-Cytarabine and at least one other anti-neoplastic agent.

The term “anti-neoplastic activity” as used herein refers to the capability of an agent to inhibit, prevent, or arrest the growth of a neoplasm or a tumor. In other words, an agent having an anti-neoplastic activity is capable of inhibiting tumor growth by at least 10%, or by at least 20%, 30%, 40%, 50%, 60%, 70%, 80%, 90%, or preferably by 100%, as compared to tumor growth in the absence of said anti-neoplastic agent.

The term “reduction in side effects” as used herein refers to the observation that a therapeutic agent or a combination of therapeutic agents is associated with fewer adverse side effects and/or less severe side effects when compared to that observed with a different therapeutic agent or combination thereof. Such a reduction in side effects may be a reduction of at least 10%, 20%, 30%, 40%, 50%, 60%, 70%, 80%, 90%, or 100%, as compared to side effects associated with a different therapeutic agent or combination thereof.

The term “epigenetic modifier” as used herein refers to an agent that affects gene expression and function by altering the chemical marking of the genome. Epigenetic marks include, for example, DNA methylation as well as methylation and acetylation of proteins associated with DNA, such as histones. The effects of epigenetic modifier do not involve changes in the DNA sequence.

The term “therapeutically effective amount” of the compound is that amount of the compound which is sufficient to provide a beneficial effect to the subject to which the compound is administered. An effective amount of the compound may vary according to factors such as the disease state, age, sex, and weight of the individual.

The terms “treatment”, “treat”, “treating” and the like, are meant to include slowing, arresting or reversing the progression of a cancer disease. These terms also include alleviating, ameliorating, attenuating, eliminating, or reducing one or more symptoms of a cancer disease, even if the disease is not actually eliminated and even if progression of the disease is not itself slowed or reversed. A subject refers to a mammal, preferably a human being.

As used herein, the term “synergy” (or “synergistic”) means that the effect achieved with the methods and combinations of this disclosure is greater than the sum of the effects that result from using the individual agents alone, e.g., using the BST-236 (or a salt thereof) alone and the at least one additional anti-neoplastic agent (e.g., azacitidine) alone. For example, the effect (e.g., apoptosis of cells, a decrease in cell viability, cytotoxicity, a decrease in cell proliferation, a decrease in cancer cell survival, inhibition of tumor growth, a reduction in tumor volume, tumor stasis, overall survival, and/or time to disease progression, etc. as described herein) achieved with the combination of a BST-236 (or a salt thereof) and the at least one additional anti-neoplastic agent (e.g., azacitidine) is about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 12 fold, about 15 fold, about 20 fold, about 25 fold, about 30 fold, about 50 fold, about 100 fold, at least about 1.2 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 8 fold, at least about 9 fold, at least about 10 fold, of the sum of the effects that result from using BST-236 (or a salt thereof) alone or the at least one additional anti-neoplastic agent (e.g., azacitidine) alone.

Synergistic effects of the combination may also be evidenced by additional, novel effects that do not occur when either agent is administered alone, or by reduction of adverse side effects when either agent is administered alone.

Methods for determining proliferation of cells (e.g., reduced proliferation) include assays for measuring cytotoxic effects of agents/compositions described herein. Cytotoxicity effects can be determined by any suitable assay, including, but not limited to, assessing cell membrane integrity (using, e.g., dyes such as trypan blue or propidium iodide, or using lactate dehydrogenase (LDH) assay), measuring enzyme activity, measuring cell adherence, measuring ATP production, measuring co-enzyme production, measuring nucleotide uptake activity, crystal violet method, Tritium-labeled Thymidine uptake method, measuring lactate dehydrogenase (LDH) activity, 3-(4,5-Dimethyl-2-thiazolyl)-2,5-diphenyl-2H-tetrazolium bromide (MTT) or MTS assay, sulforhodamine B (SRB) assay, WST assay, clonogenic assay, cell number count, monitoring cell growth, apoptosis, etc.

Apoptosis of cells may be assayed by any suitable method, including, but not limited to, TUNEL (terminal deoxynucleotidyl transferase dUTP nick end labeling) assay, assaying levels of cytochrome C release, assaying levels of cleaved/activated caspases, assaying 5-bromo-2′-deoxyuridine labeled fragmented DNA, assaying levels of survivin etc.

Other methods that can be used to show the synergistic effects of the present methods, pharmaceutical compositions and combinations include, but are not limited to, clonogenic assay (colony formation assay) to show decrease in cell survival and/or proliferation, studying tumor volume reduction in animal models (such as in mice, etc.).

A reduction in cancer burden may be determined using methods known in the art, including, without limitation, by determining the number of cancer cells in the blood and/or bone marrow, use of calipers to measure tumor size (when present) and various methods for visualizing tumor size in situ, including computer assisted tomography (CAT) scans, positron emission tomography (PET) scans, 3 dimensional sonography, x-ray, ultrasound; each of may be performed with or without contrast agents.

In one embodiment, advantageously, such synergy provides greater efficacy at the same doses or lower doses, reduced side effects, and/or prevents or delays the build-up of multi-drug resistance.

The term “about” in reference to a numerical value stated herein is to be understood as the stated value +/−10%.

The aspartic acid used in this invention is either in the L or the D configuration.

The term “pharmaceutically acceptable salt” of a drug refers to a salt according to IUPAC conventions. Pharmaceutically acceptable salt is an inactive ingredient in a salt form combined with a drug. The term “pharmaceutically acceptable salt” as used herein refers to salts of the compound (1) which are substantially non-toxic to living organisms. Typical pharmaceutically acceptable salts include those salts prepared by reaction of the compounds of the present invention with a pharmaceutically acceptable mineral, base, acid or salt. Acid salts are also known as acid addition salts (see herein below). Pharmaceutically acceptable salts are known in the art (Stahl and Wermuth, 2011, Handbook of pharmaceutical salts, Second edition).

Pharmaceutically acceptable acids which can be used for the preparation of the salts of Asp-Cytarabine include, but are not limited to, acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid and oxalic acid.

According to exemplary embodiments, the salt form of the conjugate Asp-Cytarabine is acetate or hydrochloride.

### Pharmaceutical Compositions

The present invention provides pharmaceutical compositions comprising the compound of the formula (1) and/or at least one additional anti-neoplastic agent, and a pharmaceutically acceptable carrier or diluent, optionally further comprising one or more excipients.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents.

For intravenous administration of a therapeutic compound, water is a preferred carrier. Saline solutions and aqueous dextrose and glycerol solutions can also be employed.

According to some embodiments, the composition comprising Asp-Cytarabine is formulated for intravenous administration is an aqueous isotonic solution having osmolarity of about 200-400 mOsm and a pH of 4-8. The pharmaceutically acceptable carrier of Asp-Cytarabine can be, for example, a buffered saline solution, a buffered dextrose solution, or a buffered glycerol solution having osmolarity of about 200-300 mOsm preferably of about 300 mOsm, and a pH of 4-8.

Alternatively, the buffered saline for Asp-Cytarabine composition can be, for example, Hank's balanced salt solution, Earle's balanced salt solution, Gey's balanced salt solution, HEPES buffered saline, phosphate buffered saline, Plasma-lyte, Ringer's solution, Ringer Acetate, Ringer lactate, Saline citrate, or Tris buffered saline.

The buffered dextrose solution for Asp-Cytarabine composition can be, for example, acid-citrate-dextrose solution or Elliott's B solution.

According to exemplary embodiments, the solution for injection of Asp-Cytarabine is Multiple Electrolytes Injection or Compound Sodium Lactate.

The pharmaceutical composition can further comprise pharmaceutical excipients including, but not limited to, tonicity agents such as sodium chloride, potassium chloride, magnesium chloride, sodium gluconate, sodium acetate, calcium chloride, sodium lactate, and the like. The composition, if desired, can also contain minor amounts of sugar alcohols; wetting or emulsifying agents; and pH adjusting agents; antibacterial agents such as benzyl alcohol or methyl parabens; antioxidants such as ascorbic acid or sodium bisulfite; and chelating agents such as ethylenediaminetetraacetic acid.

Pharmaceutical compositions for parenteral administration can be formulated as solutions, suspensions, emulsions, or the like, of the active compounds. Such suspensions may be prepared as oily injection suspensions or aqueous injection suspensions. For oily suspension injections, suitable lipophilic solvents or vehicles can be used including fatty oils such as sesame oil, or synthetic fatty acids esters such as ethyl oleate, triglycerides or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds, to allow for the preparation of highly concentrated solutions.

For transmucosal and transdermal administration, penetrants appropriate to the barrier to be permeated may be added to the composition. Such penetrants include, for example, DMSO, polyethylene glycol, or any penetrant known in the art.

For oral administration, the compounds can be formulated by combining the active compound with pharmaceutically acceptable carriers and excipients as known in the art. Such carriers enable the compounds of the invention to be formulated as tablets, pills, dragees, capsules, liquids, gels, syrups, slurries, suspensions, and the like, for oral ingestion by a subject. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating can be useful if it is desirable to prevent exposure of the compound of the invention to the gastric environment.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

The pharmaceutical composition comprising the anti-neoplastic agent can be formulated in a form similar to or different from the formulation of the pharmaceutical composition comprising compound (1). For example, the pharmaceutical composition of compound (1) can be formulated in a form adapted for intravenous infusion, while the pharmaceutical composition of the anti-neoplastic agent can be formulated in a form adapted for oral, subcutaneous, or intravenous administration.

The dosage of compound (1) and the dosage of the anti-neoplastic agent administered according to the method of the present invention depend on many factors including the age of the subject being treated, the stage of the cancer disease, the route of administration, and the judgment of the prescribing physician.

It should be understood that the methods of the present invention can further comprise administering one or more additional pharmaceutical compositions, each comprises a different anti-neoplastic agents, e.g., the method can include administering two, three, four or more pharmaceutical compositions, each comprises a different anti-neoplastic agent being administered prior to, concurrently with, and/or after administering the pharmaceutical composition comprising compound (1). In a particular embodiment, the one or more additional pharmaceutical compositions are administered within 4 hours of each other or within 2 hours of each other.

### Therapeutic Use

The present invention provides methods of reducing cancer cell proliferation and/or inhibiting cancer cell growth and/or methods of inhibiting cancer cell survival comprising administering to a subject: (a) a pharmaceutical composition comprising a therapeutically effective amount of the compound (1), or a pharmaceutically acceptable salt thereof, and (b) a pharmaceutical composition comprising a therapeutically effective amount of one, two or more additional anti-neoplastic agents, as described herein above.

In a particular embodiment, methods for treating a cancer in a subject afflicted with cancer are presented, comprising administering to a subject: (a) a pharmaceutical composition comprising a therapeutically effective amount of the compound (1), or a pharmaceutically acceptable salt thereof, and (b) a pharmaceutical composition comprising a therapeutically effective amount of one, two or more additional anti-neoplastic agents, as described herein above.

According to some embodiments, the cancer cell is a cancer cell of a hematological cancer or a non-hematological cancer. Thus, the methods of the present invention are useful for treating cancer selected from the group consisting of hematological cancers and non-hematological cancers.

Hematological cancers include leukemias, lymphomas, myelomas, and Myelodysplastic Syndromes (MDS) including, but not limited to, myeloid leukemia, e.g., acute myeloid leukemia (AML), chronic myeloid leukemia (CML); lymphocytic leukemia, e.g., acute lymphocytic leukemia (ALL), chronic lymphocytic leukemia (CLL); Hodgkin's lymphoma; Non-Hodgkin's lymphoma; multiple myeloma; and Waldenstrom's macroglobulinemia. Each possibility represents a separate embodiment of the invention.

The term “Myelodysplastic Syndromes” (MDS) refers to a heterogeneous group of hematopoietic disorders characterized by blood cytopenias, ineffective hematopoiesis and a hypercellular bone marrow. The MDSs are preleukemic conditions in which transformation into acute myeloid leukemia (AML) occurs in approximately 30-40% of cases. Unless allogenic stem cell transplantation can be offered, MDS is generally considered to be an uncurable condition.

Non-hematological cancers also known as solid tumors include, but are not limited to, sarcoma, carcinoma, fibrosarcoma, myxosarcoma, liposarcoma, chondrosarcoma, osteogenic sarcoma, chordoma, angiosarcoma, endotheliosarcoma, mesothelioma, Ewing's tumor leiomydsarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor cervical cancer, testicular tumor, lung carcinoma, small cell lung carcinoma, bladder carcinoma, epithelial carcinoma, astrocytoma, Kaposi's sarcoma, and melanoma. Each possibility represents a separate embodiment of the invention.

Non-hematological cancers include cancers of organs, wherein the cancer of an organ includes, but is not limited to, breast cancer, bladder cancer, colon cancer, rectal cancer, endometrial cancer, kidney cancer, lung cancer, cervical cancer, pancreatic cancer, prostate cancer, testicular cancer, thyroid cancer, ovarian cancer, brain cancer including ependymoma, glioma, glioblastoma, medulloblastoma, craneopharyngioma, pinealoma, acustic neuroma, hemangioblastoma, oligodendroglioma, menangioma, neuroblastoma, retinoblastoma, and their metastasis. Each possibility represents a separate embodiment of the invention.

The method of the present invention can be useful for treating a neoplastic disease in subjects having organ dysfunction, such as hepatic dysfunction, renal dysfunction, pancreatic dysfunction, bone marrow dysfunction, and cerebellar dysfunction.

The term “hepatic dysfunction” refers to a state in which the liver function is decreased relative to a normal state. In general, hepatic dysfunction is a state characterized in that one or more measurement values of inspection items for liver function (e.g. levels of blood AST, ALT, ALP, TTT, ZTT, total bilirubin, total protein, albumin, lactate dehydrogenase, choline esterase and the like) are deviated from the range of normal values (reference values). Hepatic dysfunction is characteristic of diseases including, but not limited to, fulminant hepatitis, chronic hepatitis, viral hepatitis, alcoholic hepatitis, hepatic fibrosis, liver cirrhosis, hepatic cancer, autoimmune hepatitis, drug allergic hepatopathy, and primary biliary cirrhosis.

Renal dysfunction is characteristic of diseases including, but not limited to, acute renal failure, glomerulonephritis, chronic renal failure, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic renal disease, interstitial nephritis, acute hemolytic uremic syndrome, medullary cystic disease, medullary sponge kidney, hereditary nephritis, and nail-patella syndrome.

Pancreatic dysfunction is characteristic of diseases including, but not limited to, diabetes, hyperglycemia, impaired glucose tolerance, and insulin resistance.

Bone marrow dysfunction is characteristic of diseases such as, for example, osteomyelitis, dyshematopoiesis, ion deficiency anemia, pernicious anemia, megaloblastosis, hemolytic anemia, and aplastic anemia.

Cerebellar dysfunction is characteristic of motor and neuro-behavioral disorders such as, for example, hypotonia, dysarthria, dysmetria, dysdiadochokinesia, impaired reflex, and intention tremor.

The pharmaceutical compositions of the invention may be administered by any suitable administration route selected from the group consisting of parenteral, oral, nasal, topical, transdermal, vaginal, and rectal administration routes. According to some embodiments, the route of administration is via parenteral administration. Parenteral route of administration includes, for example, intravenous, intraarterial, intramuscular, subcutaneous, intraperitoneal, intracerebral, intracerebroventricular, intrathecal or intradermal administration route. The pharmaceutical compositions can be administered systemically, for example, by intravenous (i.v.) or subcutaneous (s.c.) injection or infusion. According to a certain embodiment, the pharmaceutical composition comprising Asp-Cytarabine is administered by intravenous infusion for 30 minutes to 2 hours, such as for 1 hour.

Toxicity and therapeutic efficacy of the compounds described herein can be determined by standard pharmaceutical procedures in cell cultures or experimental animals, e. g., by determining the IC50 (the concentration which provides 50% inhibition of cell growth) and the MTD (Maximal tolerated dose in tested animals) for a subject compound. The data obtained from cell culture assays and animal studies can be used in formulating a range of dosages for use in human subjects. The exact formulation, route of administration and dosage can be chosen by the individual physician in view of the patient's condition (See e.g., Fingl, et al., 1975, in “The Pharmacological Basis of Therapeutics”, Ch. 1 pp. 1).

The compound (1) can be administered in a daily dose ranging from about 0.3 g/m² to about 10 g/m² of the subject's body surface area. According to some embodiments, the compound Asp-Cytarabine can be administered at a daily dose ranging from about 0.5 g/m² to about 6 g/m² of the subject's surface area. According to other embodiments, the compound, Asp- Cytarabine can be administered at a daily dose of about 0.3, 0.5, 0.8, 1, 1.5, 2, 2.3, 2.5, 3, 3.5, 4, 4.5, 5, 6, 7, 8, 9, 10 g/m² of the subject's surface area or any dose in-between.

According to some embodiments, Asp-Cytarabine is administered by intravenous infusion at a daily dose ranging from 0.3 g/m² to 6 g/m² of the subject's body surface area. In a more particular embodiment, the Asp-Cytarabine is administered by intravenous infusion at a daily dose ranging from 0.4 g/m² to 6 g/m² of the subject's body surface area. In still more particular embodiments, the Asp-Cytarabine is administered by intravenous infusion at a daily dose ranging from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; or from 1 g/m² to 6 g/m²of the subject's body surface area.

According to some embodiments, Asp-Cytarabine is administered by intravenous infusion at a daily dose ranging from 0.3 g/m² to 10 g/m² of the subject's body surface area. In further embodiments, the Asp-Cytarabine daily dose ranging from 0.3 g/m² to 10 g/m² of the subject's body surface area may be reduced by at least 10%, 20%, 30%, 40%, 50%, 60%, 70%, 80% or 90% when the Asp-Cytarabine is administered in combination therapy with at least one additional anti-neoplastic agent. A reduction in Asp-Cytarabine dose may be facilitated due to synergistic therapeutic activity observed when Asp-Cytarabine is used in combination with the at least one additional anti-neoplastic agent.

Combination therapies of the present invention for the treatment of hematological malignancies can include co-administration or sequential administration of the following anti-neoplastic drugs:

Asp-Cytarabine+azacitidine

Asp-Cytarabine+decitabine

Asp-cytarabine+guadecitabine

Asp-Cytarabine+venetoclax (ABT-199)

Asp-Cytarabine+daunorubicin/idarubicin

Asp-Cytarabine+mitoxantron

Asp-Cytarabine+midostaurin

Asp-cytarabine+crenolanib

Asp-cytarabine+gilteritinib

Asp-cytarabine+sorafenib

Asp-cytarabine+quizartinib

Asp-cytarabine+vosaroxin

Asp-cytarabine+AG221 (enasidenib)

Asp-cytarabine+AG120

Asp-cytarabine+idasanutlin

Asp-cytarabine+glasdegib

Asp-cytarabine+SL-401

Asp-cytarabine+pracinostat

Asp-cytarabine+entinostat

Asp-cytarabine+nivolumab

Asp-Cytarabine+methotrexate

Asp-Cytarabine+arsenic trioxide

Asp-Cytarabine+bevacizumab (Avastin)

Asp-Cytarabine+rituximab

Asp-Cytarabine+interferon

Asp-Cytarabine+imatinib

Asp-Cytarabine+daunorubicin+midostaurin

Asp-cytarabine+daunorubicin+crenolanib

Asp-cytarabine+daunorubicin+quizartinib

Asp-cytarabine+daunorubicin+gilteritinib

Asp-cytarabine+daunorubicin+sorafenib

Asp-cytarabine+hydroxyurea+azacitidine

Asp-Cytarabine+daunorubicin+all trans retinoic acid

Asp-Cytarabine+daunorubicin+mitoxantrone

Asp-Cytarabine+daunorubicin+cladribine

Asp-Cytarabine+idarubicin+mitoxantrone

Asp-Cytarabine+methotrexate+corticosteroid(s)

Asp-Cytarabine+methotrexate+rituximab

Asp-Cytarabine+methotrexate+mercaptopurine

Asp-Cytarabine+mitoxantrone+etoposide

Asp-Cytarabine+etoposide+rituximab

Asp-Cytarabine+mitoxantrone+cladribine+G-CSF

Asp-Cytarabine+mitoxantrone+etoposide+G-CSF

Asp-Cytarabine+idarubicin+fludarabine+topotecan

Asp-Cytarabine+dexamethasone+cisplatin+rituximab.

Therapeutically effective doses and administration regimen of some of the anti-neoplastic agent are exemplified as follows: hydroxyurea can be administered orally at a daily dose of 0.5 g to 1 g, azacitidine can be administered intravenously or subcutaneously at a daily dose of 75 mg/m² for 7 days, daunorubicin can be administered at a daily dose of 60 mg/m² to 90 mg/m² for 3 days, midostaurin can be administered at a daily dose of 50 mg/m² for 14 days, idarubicin can be administered at a daily dose of 10 mg/m² to 12 mg/m², all-trans retinoic acid can be administered at a daily dose of 45 mg/m² for 15 days, mitoxantrone can be administered at a daily dose of 10 mg/m² to 12 mg/m² for 3 days, and etoposide can be administered at a daily dose of 50 mg/m² for 5 days to 70 mg/m² for 7 days.

In particular embodiments of combination therapy/treatment described herein, the at least one additional anti-neoplastic agent may be selected from the list of agents presented in Table 1 and dosed within indicated ranges as follows:

| | Dose in clinical | |
|---|---|---|
| Agent/Compound | study/market | Company |
| Azacytidine | 75 mg-100 mg/m²/day | Celgene |
| decitabine | 15 mg/m² for 3 h | (generic) - Sandoz, |
| | every 8 h or 20 mg/m² | Dr. Reddy's etc |
| | for 1 h once daily (IV) | |
| guadecitabine (SGI- | 60 mg/m² (SC) | Astex Pharmaceuticals |
| 110) | | |
| gemcitabine | 1000 mg/m² (IV) | (generic) - Lilly, Teva etc |
| zidovudine | 100-600 mg/day (ora) | GSK, Mylan, Cipla |
| | | (generic) |
| venetoclax | 20-600 mg/day (oral) | Abbvie |
| sorafenib | 200-800 mg/day (oral) | Bayer, Mylan |
| | | (generic) |
| midostaurin | 100-200 mg/day (oral) | Novartis |
| quizartinib | 20-30 mg/day (oral) | Daiichi Sankyo |
| crenolanib | 300 mg/day (oral) | Arog pharmaceuticals |
| gilertinib | 120 mg/day (oral) | Astellas |
| daunorubicin | 75 mg-100 mg/m² | (generic) West ward, |
| | (IV) | Teva etc |
| idarubicin | 5 mg-12 mg/m² (IV) | (generic) Teva, West |
| | | ward etc |
| doxorubicin | 40 mg-75 mg/m² (IV) | (generic) Teva, West |
| | | ward etc |
| AG-120 (ivosidenib) | 500 mg/day (oral) | Agios |
| AG-221 (enasidenib, | 100 mg/day (oral) | Celgene/Agios |
| IDHIFA) | | |
| IDH-305 | 100-900 mg/day (oral) | Novartis |
| FT-2102 | 150-300 mg/day (oral) | Forma therapeutics |

Also indicated in Table 1 are companies from which the particular agent/compound may be purchased. Each of the above agents/compounds are commercially available from at least the provider/company indicated above.

In more particular embodiments, the at least one additional anti-neoplastic agent may be dosed at a lower range when administered in combination therapy with Asp-Cytarabine. Accordingly, the doses indicated in Table 1 may be reduced by at least 10%, 20%, 30%, 40%, 50%, 60%, 70%, 80% or 90%.

Additionally or alternatively, uses and methods pertaining to combination therapy with Asp-Cytarabine and calling for the above doses listed in Table 1 and each of the above % reduced doses for each of the anti-neoplastic agents may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m²of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the additional anti-neoplastic agent are used concurrently or sequentially; and/or where used sequentially, the Asp-cytarabine may be used/administered before or after the additional anti-neoplastic agent.

It is noteworthy that PK analysis from Phase I/II studies revealed that following infusion cessation, concentrations of BST-236 rapidly declined in an apparent biphasic manner, reaching ≤5% of the peak (although still detectable) at 6-10 hours from the completion of the infusion. Accordingly, synergistic interactions may be observed within 6-10 of administration of BST-236.

In an embodiment wherein Asp-Cytarabine (BST-236) is used in combination with a pyrimidine analog (e.g., at least one of azacitidine, decitabine, guadecitabine (SGI-110), gemcitabine, or zidovudine), wherein at least one of azacitidine is dosed at 75 mg-100 mg/m²/day intravenously or subcutaneously for 7 days, decitabine is dosed at 15 mg/m² for 3 h every 8 h or 20 mg/m² for 1 h once daily (IV), guadecitabine (SGI-110) is dosed at 60 mg/m2 (SC), gemcitabine is dosed at 1000 mg/m² (IV), or zidovudine is dosed at 100-600 mg/day (oral) in uses and methods described herein or each of these respective dose ranges may be reduced by at least 10%, 20%, 30%, 40%, 50%, 60%, 70%, 80% or 90% and additionally or alternatively uses and methods calling for such pyrimidine analogs may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m² of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the pyrimidine analog are used concurrently or sequentially; and/or where used sequentially, the Asp-cytarabine may be used/administered before or after the pyrimidine analog.

In an embodiment wherein Asp-Cytarabine (BST-236) is used in combination with a BCl-2 inhibitor (e.g., at least one of venetoclax), wherein, e.g., venetoclax is dosed at 20-600 mg/day (oral) in uses and methods described herein or wherein this dose range may be reduced by at least 10%, 20%, 30%, 40%, 50%, 60%, 70%, 80% or 90% and additionally or alternatively uses and methods calling for such a BCl-2 inhibitor may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m² of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the BCl-2 inhibitor are used concurrently or sequentially; and/or where used sequentially, the Asp-cytarabine may be used/administered before or after the BCl-2 inhibitor.

In an embodiment wherein Asp-Cytarabine (BST-236) is used in combination with a kinase inhibitor (e.g., at least one kinase inhibitor comprising, e.g., Sorafenib), wherein at least one of Sorafenib is dosed at 200-800 mg/day (oral) in uses and methods described herein or this respective dose range may be reduced by at least 10%, 20%, 30%, 40%, 50%, 60%, 70%, 80% or 90% and additionally or alternatively uses and methods calling for such kinase inhibitors may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m² of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the kinase inhibitor are used concurrently or sequentially; and/or where used sequentially, the Asp-cytarabine may be used/administered before or after the kinase inhibitor.

In an embodiment wherein Asp-Cytarabine (BST-236) is used in combination with a FLT3 inhibitor (e.g., at least one FLT3 inhibitor comprising, e.g., midostaurin, gilteritinib, quizartinib, or crenolanib), wherein at least one of midostaurin is dosed at 100-200 mg/day (oral), gilteritinib is dosed at 120 mg/day (oral), quizartinib is dosed at 20-30 mg/day (oral), or crenolanib is dosed at 300 mg/day (oral) in uses and methods described herein or each of these respective dose ranges may be reduced by at least 10%, 20%, 30%, 40%, 50%, 60%, 70%, 80% or 90% and additionally or alternatively uses and methods calling for such FLT3 inhibitors may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m² of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the FLT3 inhibitor are used concurrently or sequentially; and/or where used sequentially, the Asp-cytarabine may be used/administered before or after the FLT3 inhibitor.

In an embodiment wherein Asp-Cytarabine (BST-236) is used in combination with an anthracycline (at least one anthracycline comprising, e.g., daunorubicin, idarorubicin, or doxorubicin), wherein at least one of daunorubicin is dosed at 75 mg-100 mg/m² (IV), idarorubicin is dosed at 5 mg-12 mg/m² (IV), or doxorubicin is dosed at 40 mg-75 mg/m² (IV) in uses and methods described herein or each of these respective dose ranges may be reduced by at least 10%, 20%, 30%, 40%, 50%, 60%, 70%, 80% or 90% and additionally or alternatively uses and methods calling for such anthracyclines may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m² of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the anthracycline are used concurrently or sequentially; and/or where used sequentially, the Asp-cytarabine may be used/administered before or after the anthracycline.

In an embodiment wherein Asp-Cytarabine (BST-236) is used in combination with an IDH inhibitor (at least one IDH inhibitor comprising, e.g., AG-120 (ivosidenib), AG-221 (enasidenib, IDHIFA), IDH-305, or FT-2102), wherein at least one of AG-120 (ivosidenib) is dosed at 500 mg/day (oral), AG-221 is dosed at 100 mg/day (oral), IDH-305 is dosed at 100-900 mg/day (oral), or FT-2102 is dosed at 150-300 mg/day (oral) in uses and methods described herein or each of these respective dose ranges may be reduced by at least 10%, 20%, 30%, 40%, 50%, 60%, 70%, 80% or 90% and additionally or alternatively uses and methods calling for such IDH inhibitors may include one or more of the following features individually or in combination: wherein Asp-Cytarabine is administered parenterally (e.g., by intravenous infusion) at a daily dose ranging from 0.3 g/m² to 6 g/m² of the subject's body surface area; from 0.4 g/m² to 6 g/m² of the subject's body surface area; from 0.5 g/m² to 6 g/m² of the subject's body surface area; from 0.6 g/m² to 6 g/m² of the subject's body surface area; from 0.7 g/m² to 6 g/m² of the subject's body surface area; from 0.8 g/m² to 6 g/m² of the subject's body surface area; from 0.9 g/m² to 6 g/m² of the subject's body surface area; from 1 g/m² to 6 g/m² of the subject's body surface area; from 0.3 g/m² to 10 g/m² of the subject's body surface area; wherein a range from 0.3 g/m² to 6 g/m² or a range of 0.3 g/m² to 10 g/m² of the subject's body surface area is reduced by at least 10%, 20%, 30%, 40%, 50%, 60%, 70%, 80% or 90%; and/or wherein the Asp-Cytarabine and the IDH inhibitor are used concurrently or sequentially; and/or where used sequentially, the Asp-cytarabine may be used/administered before or after the IDH inhibitor.

According to some embodiments, the pharmaceutical composition comprising compound (1), the pharmaceutical composition comprising the anti-neoplastic agent or the combination thereof, is administered at least once a month. According to additional embodiments, the pharmaceutical compositions are administered at least twice a month. According to further embodiments, the pharmaceutical compositions are administered at least once a week. According to yet further embodiments, the pharmaceutical compositions are administered at least twice a week. According to still further embodiments, the pharmaceutical compositions are administered once a day for at least one week. According to further embodiments, the pharmaceutical compositions are administered at least once a day for at least one week or until the subject is cured or in remission.

According to some embodiments, the pharmaceutical compositions can be administered once a day for at least 2, 3, 4, 5, 6, 8, 10, 12, or at least 15 consecutive days once a month. Alternatively, the pharmaceutical compositions can be administered once a day for at least 2, 3, 4, 5, 6, or 15 days twice a month, or further alternatively the pharmaceutical compositions can be administered every day or twice a week until the patient is cured or in remission.

In some embodiments, where the pharmaceutical composition is used for preventing recurrence of cancer, the pharmaceutical composition can be administered regularly for prolonged periods of time according to the clinician's instructions.

In some embodiments it may be advantageous to administer a large loading dose followed by periodic (e.g., weekly) maintenance doses over the treatment period. The compounds can also be delivered by slow-release delivery systems, pumps, and other known delivery systems for continuous infusion. Dosing regimens may be varied to provide the desired circulating levels of a particular compound based on its pharmacokinetics. Thus, doses are calculated so that the desired circulating level of a therapeutic agent is maintained.

Typically, the effective dose is determined by the activity and efficacy of the compound and the condition of the subject as well as the body weight or surface area of the subject to be treated. The dose and the dosing regimen are also determined by the existence, nature, and extent of any adverse event that accompanies the administration of the compound in a particular subject.

The following examples are to be considered merely as illustrative and non-limiting in nature. It will be apparent to one skilled in the art to which the present invention pertains that many modifications, permutations, and variations may be made without departing from the scope of the invention.

### EXAMPLE 1

### Effect of Asp-Cytarabine/BST-236 and Azacitidine (Vidaza) on Proliferation and Survival of U937 Cells

U937 human hematological cancer cells were cultured in RPMI supplemented with 10% FCS. The cells were seeded at 1×10⁵ cells/well in a total volume of 250 μl in a 96-well plate. Azacitidine (AZA) was added to the cell cultures at 5 different concentrations: 0, 100, 250, 1000, 5000 nM. Asp-Cytarabine, also designated herein below BST-236, was added to the culture at a concentration of 250 nM. All groups were analyzed in triplicates. After 72 hours of incubation at 37° C. with 5% CO₂, the cells were collected, stained with propidium iodide (PI), and immediately read by FACS. The number and percentage of viable (PI-negative) cells and the number and percentage of dead (PI-positive) cells in the culture were determined by FACScalibur using CellQuest software. The percentage of inhibition was calculated.

TABLE 2
Percentage of growth inhibition of U937 cells treated with
Asp-Cytarabine, Azacitidine and the combination thereof.

| | | | | AZA | AZA |
|---|---|---|---|---|---|
| | BST236 | AZA | AZA | (1000 | (5000 |
| Treatment | (250 nM) | (100 nM) | (250 nM) | nM) | nM) |
| % inhibition | 19.1 | 1.68 | −3.60 | 0.60 | 4.30 |
| AZA + BST236 | — | 19.94 | 34.43 | 28.65 | 49.27 |

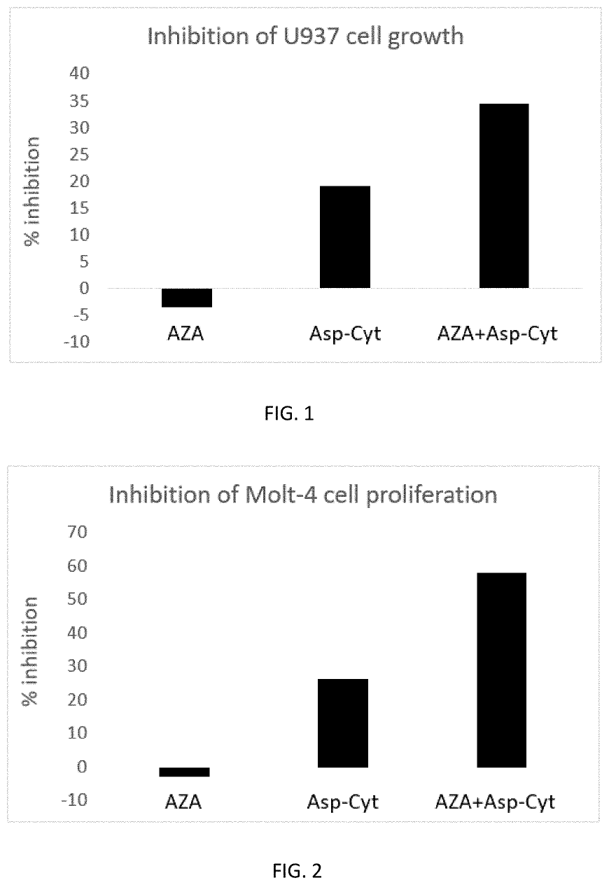

As shown in FIG. 1 and Table 2, the combined treatment of human hematological cancer cells with Asp-Cytarabine and Azacitidine resulted in a pronounced synergistic inhibition of the proliferation and survival of the hematological cancer cells.

The synergistic nature of the combination of Asp-Cytarabine and Azacytidine is underscored by results presented in Table 3 which show that a 250 nM concentration of Asp-Cytarabine is well below that determined to provide maximal inhibition of U937 cells. Indeed, 250 nM Asp-Cytarabine consistently confers less than 20% inhibition of U937 cells when administered alone. Results presented in FIG. 1 and Table 2, depicting experiments which were performed with 250 nM Asp-Cytarabine, therefore, reveal that the presence of low levels of Asp-Cytarabine synergize in a therapeutically demonstrable manner with Azacytidine at a variety of concentrations.

TABLE 3
Activity of BST-236 on U937 cells

| 48 hr | | | | | | | | |
|---|---|---|---|---|---|---|---|---|
| Treatment | 0 | 1 nM | 10 nM | 50 nM | 100 nM | 250 nM | 1000 nM | 5000 nM |
| BST236 | 16581.67 | 16958 | 16994 | 15769.67 | 15228.33333 | 14819.67 | 6185.667 | 2116 |
| 10% FCS | | | | | | | | |
| | | −2.27 | −2.49 | 4.90 | 8.16 | 10.63 | 62.70 | 87.24 |

### EXAMPLE 2

### Effect of Asp-Cytarabine and Azacitidine on Molt-4 Cell Proliferation

Molt-4 human leukemia cell line was obtained from ATCC. The cells were grown in RPMI medium containing 10% FBS and 1% glutamine Cells were seeded in 96-well plates, 50,000 cells/ml, 0.2 ml per well. Test substances were diluted in PBS and added in final concentrations of 0.1 nM to 10 μM, in a volume of 20 μl. The study was conducted in triplicates. PBS was used as a control. Plates were incubated for 72 hr at 37° C. with 5% CO₂. At the end of the treatment period, a MTT assay using the MTT reagent [3-(4,5-dimethylthiazol-2-yl)-2,5-diphenyltetrazolium bromide] was performed. MTT was added to each well at a concentration of 5 mg/ml in a volume of 0.02 ml. Plates were incubated at 37° C. for 3 hours. The plates were centrifuged at 3500 rpm for 5 minutes and the supernatant was aspirated. The pellets which contained MTT crystals were each dissolved in 0.2 ml DMSO. Absorbance was determined using ELISA reader at a wavelength of 570 nm.

As shown in FIG. 2, the combined treatment of human leukemia cells with Asp-Cytarabine and Azacitidine, each at a concentration of 8 nM, resulted in a pronounced synergistic inhibition of the proliferation of human leukemia cells.

Table 4 summarizes the IC₅₀ values for Asp-Cytarabine, Azacitidine, and the combination thereof on Molt-4 cell proliferation as obtained in the above experiment.

TABLE 4
IC₅₀ values of Asp-Cytarabine, Azacitidine, and the
combination of both on Molt-4 cell proliferation.

| Treatment | IC50 (nM) |
|---|---|
| Azacitidine | 2111 |
| Asp-Cytarabine | 47 |
| Asp-Cytarabine + Azacitidine | 11 |

The synergistic nature of the combination of Asp-Cytarabine and Azacytidine is underscored by results presented in Table 5, which show that a 10 nM concentration of Asp-Cytarabine is well below that determined to provide maximal inhibition of Molt-4 cells. Indeed, 10 nM Asp-Cytarabine consistently confers less than 10% inhibition of Molt-4 cells. In that the IC₅₀ values were determined with only 8 nM of Asp-Cytarabine in combination with Azacitidine, these results demonstrate that when used in combination, Asp-Cytarabine can be administered at lower doses and will still exhibit synergism with Azacitidine. The combination therapy, therefore, provides for improved efficacy while reducing adverse side effects that may arise from treatment protocols calling for higher dosing.

TABLE 5
Activity of Asp-Cytarabine/BST-236 on Molt-4 cells

| 48 hr | | | | | | | | |
|---|---|---|---|---|---|---|---|---|
| Treatment | 0 | 1 nM | 10 nM | 50 nM | 100 nM | 250 nM | 1000 nM | 5000 nM |
| BST236 | 19138.00 | 18778.00 | 17759.00 | 14258.67 | 12006.67 | 9572.00 | 3156.33 | 2037.33 |
| 10% FCS | | | | | | | | |
| | | 1.88 | 7.21 | 25.50 | 37.26 | 49.98 | 83.51 | 89.35 |

### EXAMPLE 3

### Effect of Asp-Cytarabine and ABT-199 (Venetoclax) on Proliferation and Survival of U937 Cells

U937 cells were cultured in RPMI supplemented with 10% FCS and seeded at 1×10⁵ cells/well in a total volume of 250 μl in a 96-well plate. ABT-199 was added to the cell cultures at 3 different concentrations: 0, 250, and 1000 nM. Asp-Cytarabine was added to the culture at a concentration of 250 nM. All groups were analyzed in triplicates. After 24 hours of incubation at 37° C. with 5% CO₂, the cells were collected and stained with propidium iodide (PI) and immediately read by FACS. The number and percentage of viable (PI-negative) cells and the number and percentage of dead (PI-positive) cells in the culture were determined by FACScalibur using CellQuest software. The percentage of inhibition was calculated.

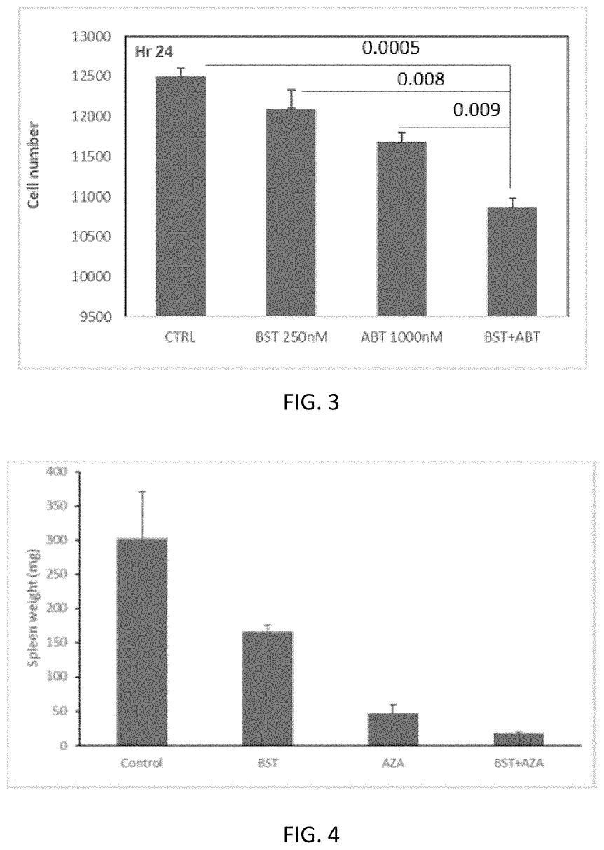

As shown in FIG. 3, the combined treatment of human hematological cancer cells with Asp-Cytarabine and ABT-199 for 24 hours resulted in a significant inhibition of the proliferation and survival of U937 cells.

In further experiments, U937 cells will be cultured in RPMI supplemented with 10% FCS and seeded at 1×10⁵ cells/well in a total volume of 250 μl in a 96-well plate. ABT-199 may be added to the cell cultures at, e.g., 6 different concentrations: 0, 10, 100, 250, 1000, and 3000 nM. Asp-Cytarabine may be added to the culture at a concentration of 250 nM. ABT-199 at these concentrations will be added 24 h before the addition of Asp-cytarabine, 12 h before the addition of Asp-cytarabine, at the same time as Asp-cytarabine and/or 12 h after the addition of Asp-cytarabine. After 24 or 48 hours of incubation at 37° C. with 5% CO₂, the cells may be collected and counted either using a coulter counter or acceptable staining methods. The percentage of inhibition will be calculated based on relative numbers of viable or dead cells relative to the total number of cells.

### EXAMPLE 4

### Effect of BST-236 in Combination with Azacytidine in an Animal Model of Leukemia

The effect of BST-236 in combination with azacytidine on the survival of U937 leukemia cells in vivo was next examined NOD scid gamma (NSG) mice were irradiated with 200 rad, 24 hours before injection with U937 cells. On Day 0, mice (4-5 animals per group), were injected intravenously (IV) with 7×10⁶ U937 cells in a total volume of 200 μL PBS. On Days 16-22 (7 days, first study) or 13-18 (6 days, second study), mice were daily injected subcutaneously (s.c.) with BST-236 (5 mg/mouse; ˜250 mg/kg), azacytidine (designated AZA; 6 mg/kg), or BST-236 (5 mg/mouse; ˜250 mg/kg) and AZA (6 mg/kg). Twenty-four hours after the last injection, mice were sacrificed, and spleen, blood, and bone marrow were analyzed for mouse and human CD45⁺ cells using fluorescence-activated cell sorting (FACS).

The results showed low levels of normal murine white blood cells (WBC) in blood, spleen and bone marrow, leading to mortality, thus indicating that the NSG mice developed leukemia following injection of U937 cells.

In the first study, all mice of the control group (n=5) and one animal (1/4) of the AZA-treated group died before the scheduled sacrifice on Day 23. All animals treated with BST-236 (4/4) and the combination of BST-236+AZA (5/5) survived until the scheduled sacrifice. After sacrifice, spleen weight, the number of human leukemia CD+45 cells and the number of murine CD+45 cells in the blood and in the spleen were examined Blood samples were not available for the 5 control mice that died prior to sacrifice.

As shown in FIG. 4, the spleens of the control mice were significantly larger than the spleens of the treated animals. Each of the treatments: either BST-236 or AZA, resulted in a reduced spleen size compared to the control mice. However, the combination of BST-236 and AZA had the greatest effect on reducing spleen weight. Indeed, the combination of BST-236 and AZA demonstrated a synergistic effect in reducing spleen size, which is an indicator of a reduction in the number of U937 cancer cells in the spleen.

### EXAMPLE 5

### Effect of BST-236 in Combination with Azacytidine in an Animal Model of Leukemia

An additional experiment in NSG mice revealed similar activity when BST-236 was dosed at 1.7 mg/mouse (˜85 mg/kg) as compared to dosing at 5 mg/mouse (˜250 mg/kg; as shown in Example 4). Given that finding, experiments will be performed using even lower doses of BST-236 to evaluate synergistic activity with, e.g., AZA. It is, for example, expected that 20 mg/kg BST-236 will be efficacious. Accordingly, in a particular embodiment, NSG mice will be dosed with 20 mg/kg BST-236 alone, 6 mg/kg AZA alone, or a combination of 20 mg/kg BST-236 and 6 mg/kg AZA to investigate synergistic activity with this dosing regimen. It is understood that synergistic activity may also be observed at lower doses of AZA in combination with 20 mg/kg BST-236.

### EXAMPLE 6

### Clinical Study of a Combination Therapy of BST-236 and AZA

A clinical study will be conducted to evaluate the performance and safety of BST-236 in combination with azacitidine in AML and ALL patients.

### Study Design

Phase I/IIa, open-label, uncontrolled, single-center study will enroll patients of 18 or more years of age with relapsed or refractory acute leukemia or those unfit for intensive therapy, as judged by the treating physician.

Patients of any age will be enrolled into 4 BST-236 escalating dose cohorts, each including 3 patients. BST-236 will be administered as a 1-hour single daily infusion for 6 consecutive days.

BST-236 doses: 1.5 g/m², 3 g/m², 4.5 g/m², and 6 g/m²

AZA doses 50-75 mg/m² daily for 7 days by injection or infusion (intravenous or subcutaneous administration)

Treatment with either of BST-236 or AZA or a combination thereof will start on the same day.

Patient response will be determined based on a variety of parameters including, without limitation, tolerability of the combination, safety of the combination (hematological and non hematological adverse events), reduction in number of circulating AML or ALL cancer cells and in the number of AML or ALL cancer cells in the bone marrow.

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

What is claimed is:

1. A method for reducing cancer cell proliferation or treating a cancer in a subject afflicted with the cancer in a subject afflicted with a cancer, comprising:
(a) administering a first pharmaceutical composition comprising a therapeutically effective amount of a compound represented by the structure of formula (1): 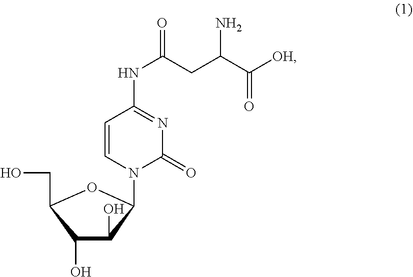
or a pharmaceutically acceptable salt thereof; and
(b) administering a second pharmaceutical composition comprising a therapeutically effective amount of at least one additional anti-neoplastic agent;
wherein the first and second pharmaceutical compositions are administered to the subject concurrently or sequentially, thereby reducing cancer cell proliferation in the subject.

2. The method of claim 1, wherein the second pharmaceutical composition is administered prior to, concomitant with, or after the first pharmaceutical composition is administered.

3. The method of claim 1, wherein the second pharmaceutical composition is administered concurrently with the first pharmaceutical composition, or within four hours from each other.

4. The method of claim 1, wherein the pharmaceutically acceptable salt of the compound of formula (1) is a salt of an organic or inorganic acid selected from acetic acid, hydrochloric acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, maleic acid, formic acid, malonic acid, nicotinic acid or oxalic acid.

5. The method of claim 4, wherein the pharmaceutically acceptable salt is a salt of acetic acid.

6. The method of claim 4, wherein the pharmaceutically acceptable salt of the compound of formula (1) is a salt of hydrochloric acid.

7. The method of claim 1, wherein the at least one anti-neoplastic agent is a pyrimidine analog, a fms like kinase-3 (FLT-3) inhibitor, a Bcl-2 inhibitor, a sonic hedgehog inhibitor, an antibody, an anthracycline, a drug that target P53, or an isocitrate dehydrogensase (IDH) inhibitor.

8. The method of claim 7, wherein the antibody is selected from the group consisting of anti CD19 antibodies, anti CD20 antibodies, anti CD22 antibodies, anti CD30 antibodies, anti CD33 antibodies, anti CD37 antibodies, anti CD38 antibodies, anti CD47 antibodies, anti CD52 antibodies, anti CD70 antibodies, anti CD79 antibodies, anti CD80 antibodies, anti CD123 (IL3) antibodies, immune checkpoint inhibitors, anti CXCR antibodies, anti growth factor antibodies or growth factor receptor antibodies, anti-metalloproteinase antibodies, anti-selectin antibodies, and antibody-drug conjugates.

9. The method of claim 8, wherein the anti CD33 antibody is gemtuzumab-ozogamicin.

10. The method of claim 8, wherein the anti CD123 antibody is CSL362, talacotuzumab or IMGN632.

11. The method of claim 8, wherein the anti CD47 antibody is Hu5F9, or CC-90002.

12. The method of claim 8, wherein the anti CD70 antibody is Argx-110.

13. The method of claim 7, wherein the sonic hedgehog is glasdegib.

14. The method of claim 7, wherein the drug that targets P53 is APR246.

15. The method of claim 7, wherein the pyrimidine analog is azacitidine, decitabine, guadecitabine (SGI-110), gemcitabine, or zidovudine.

16. The method of claim 15, wherein the pyrimidine analog is azacitidine.

17. The method of claim 7, wherein the Bcl-2 inhibitor is venetoclax (ABT-199).

18. The method of claim 7, wherein the FLT-3 inhibitor is sorafenib, midostaurin, quizartinib, crenolanib, or gilertinib.

19. The method of claim 7, wherein the anthracycline is daunorubicin, idarubicin, or doxorubicin.

20. The method of claim 7, wherein the IDH inhibitor is an IDH1 inhibitor, an IDH2 inhibitor, AG-120 (ivosidenib), AG221 (enasidenib), IDH305, or FT-2102.

21. The method of claim 1, wherein the anti-neoplastic agent is bound or attached to immune cells capable of inhibiting cancer cell growth, wherein the immune cells are chimeric antigen receptor T cells (CART).

22. The method of claim 21, wherein the CART is selected from CART123, CART33, CART34, CART38, CART56 and CART117.

23. The method of claim 1, wherein the cancer is a hematological cancer or a non-hematological cancer.

24. The method of claim 23, wherein the hematological cancer is a leukemia, a lymphoma, a myeloma or a Myelodysplastic Syndrome (MDS).

25. The method of claim 24, wherein the leukemia is Acute Myeloid Leukemia (AML), Acute Lymphoblastic Leukemia (ALL), Chronic Myeloid Leukemia (CML), or Chronic Lymphoblastic Leukemia (CLL).

26. The method of claim 25, wherein the AML is newly diagnosed AML, secondary AML, or relapsed/refractory AML.

27. The method of claim 24, wherein the lymphoma is Hodgkin's lymphoma or non-Hodgkin's lymphoma.

28. The method of claim 1, wherein the subject is a human.

29. The method of claim 28, wherein the human is a medically compromised human.

30. The method of claim 29, wherein the medically compromised human is an elderly human, a human having hepatic dysfunction, a human having renal dysfunction, a human having pancreatic dysfunction, a human having bone marrow dysfunction, a human having cerebellar dysfunction, a human having an immunological disorder, a human having refractory or relapsed hematological cancer, or any combination thereof.

31. The method of claim 30, wherein the elderly human is 70 or more years of age.

32. The method of claim 1, wherein the pharmaceutical composition comprising the compound of formula (1) is administered parenterally.

33. The method of claim 1, wherein the first pharmaceutical composition is administered intravenously.

34. The method of claim 1, wherein the dosage of the compound of formula (1) administered to the subject ranges from about 0.3 g/m² to about 6 g/m² of the subject's body surface area per day.

35. The method of claim 1, wherein the dosage of the compound of formula (1) administered to the subject ranges from about 0.8 g/m² to about 6 g/m² of the subject's body surface area per day.

36. A method for reducing cancer cell proliferation or treating cancer in a subject afflicted with the cancer, comprising:
(a) administering a first pharmaceutical composition comprising a therapeutically effective amount of a compound represented by the structure of formula (1): 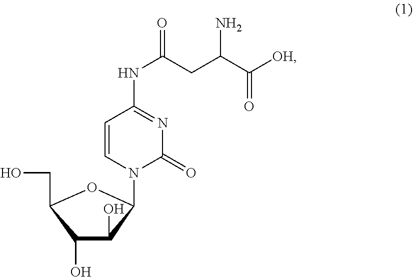
or a pharmaceutically acceptable salt thereof, and
(b) administering a therapeutically effective amount of a second pharmaceutical composition comprising at least one additional anti-neoplastic agen, wherein the at least one anti-neoplastic agent is a pyrimidine analog, a fms like kinase-3 (FLT-3) inhibitor, a sonic hedgehog inhibitor, an antibody, a drug that target P53 a Bcl-2 inhibitor, an anthracycline, or an isocitrate dehydrogensase (IDH) inhibitor,
wherein the first and second pharmaceutical compositions are administered to the subject concurrently or within four hours of each other, thereby reducing cancer cell proliferation in the subject; and
wherein the administering results in a reduction in side effects in the subject, wherein the side effects comprise at least one of mucositis, diarrhea, or alopecia, relative to side effects observed in subjects treated with cytarabine and the at least one additional anti-neoplastic agent or a second pharmaceutical composition comprising cytarabine and the at least one additional anti-neoplastic agent.

37. A method for reducing cancer cell proliferation or treating cancer in a subject afflicted with the cancer, comprising administering a pharmaceutical composition-comprising:
(i) a therapeutically effective amount of a compound represented by the structure of formula (1): 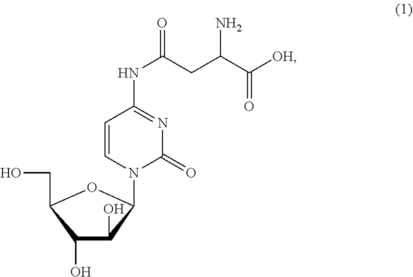
or a pharmaceutically acceptable salt thereof;
(ii) a therapeutically effective amount of an additional anti-neoplastic agent, wherein the at least one anti-neoplastic agent is a pyrimidine analog, a FLT-3 inhibitor, a Bcl-2 inhibitor, an anthracycline, a sonic hedgehog inhibitor, an antibody, a drug that target P53 or an isocitrate dehydrogensase (IDH) inhibitor; and
(iii) a pharmaceutically acceptable excipient.
